(19) United States
(12) Patent Application Publication
(10) Pub. No.: US 2015/0212064 A1
(43) Pub. Date: Jul. 30, 2015

(54) DEGRANULATION INDICATOR AND METHODS OF USE THEREOF

(71) Applicants: Jordan S. Orange, Houston, TX (US); Gregory D. Rak, Philadelphia, PA (US)

(72) Inventors: Jordan S. Orange, Houston, TX (US); Gregory D. Rak, Philadelphia, PA (US)

(73) Assignee: THE CHILDRENS'S HOSPITAL OF PHILADELPHIA, Philadelphia, PA (US)

(21) Appl. No.: 14/238,583

(22) PCT Filed: Aug. 13, 2012
(86) PCT No.: PCT/US12/50557

§ 371 Date: Oct. 3, 2014

Related U.S. Application Data
(60) Provisional application No. 61/523,038, filed on Aug. 12, 2011.

(51) Int. Cl. G01N 33/50; C07K 14/705; C07K 14/435; C07K 14/715

(52) U.S. Cl. CPC G01N 33/505; C07K 14/7155; C07K 14/70596; C07K 14/43595; G01N 2458/30; C07K 2319/02; C07K 2319/03; C07K 2319/06

(57) ABSTRACT

Compositions and methods for assessing degranulation in NK cells are provided.

This application claims priority under 35 U.S.C. §119(e) to U.S. Provisional Patent Application No. 61/523,038, filed Aug. 12, 2011. The foregoing application is incorporated by reference herein.

Pursuant to 35 U.S.C. §202(c) it is acknowledged that the U.S. Government has rights in the invention described, which was made with funds from the National Institutes of Health, Grant Numbers AI67946, GM070898, RR22482 and T32 AR07442.

### FIELD OF THE INVENTION

The present invention relates to the fields of cell based assays and degranulation events in lymphocytes. More specifically, the invention provides compositions and methods for visualizing these events in real time that are useful for the identification of agents which modulate this process.

### BACKGROUND OF THE INVENTION

Several publications and patent documents are cited throughout the specification in order to describe the state of the art to which this invention pertains. Each of these citations is incorporated herein by reference as though set forth in full.

Natural killer (NK) cells are lymphocytes of the innate immune system that function in clearance of tumor and virally infected cells. Elimination of susceptible target cells is tightly regulated and follows ligation of germline-encoded activation receptors. As NK cells do not require receptor gene rearrangement, they are constitutively enabled for cytotoxicity. Thus, NK cell activation must be tightly regulated to ensure that healthy cells remain unharmed. Efficient lysis requires the tight adherent formation between the NK cell and the target cell termed the immunologic synapse (IS). The formation of a mature, cytolytic synapse between an NK cell and a target cell occurs in stages that can be thought of as checkpoints in the activation process (Wulfing et al. (2003) Proc. Natl. Acad. Sci., 100:7767-72; Orange, J. S. (2008) Nat. Rev. Immunol., 8:713-25; Orange et al. (2003) Proc. Natl. Acad. Sci., 100:14151-6). Major cytoskeletal steps that are required in this process include the rearrangement of filamentous actin (F-actin) and the polarization of the microtubule organizing center (Katz et al. (1982) J. Immunol., 129:2816-25; Carpen et al. (1983) J. Immunol., 131:2695-8; Kupfer et al. (1983) Proc. Natl. Acad. Sci., 80:7224-8). These events culminate in the directed secretion of lytic granule contents at the IS, which is prerequisite for NK cell cytotoxocity.

To date effective biochemical tools have not been available to assess the complex degranulation reactions described above. Improved reagents and methods for this purpose are needed to identify agents which modulate this process, thereby providing new therapeutics for the treatment of disease.

### SUMMARY OF THE INVENTION

In accordance with the present invention, isolated degranulation indicator fusion proteins are provided. In a particular embodiment, the degranulation indicator comprises at least one pH sensitive detectable agent; at least one transmembrane domain; at least one lysosome targeting moiety; and, optionally, at least one endoplasmic reticulum targeting signal sequence. The degranulation indicator may further comprise linkers between the above domains. In a particular embodiment, the endoplasmic reticulum targeting signal sequence and/or the transmembrane domain is derived from the IL-2 receptor α chain. In a particular embodiment, the lysosome targeting moiety is the cytoplasmic tail of the lysosomal-associated membrane protein 1 (LAMP1). The pH sensitive detectable agent may be a fluorescent protein such as pHluorin.

In accordance with another aspect of the instant invention, nucleic acid molecules encoding at least one degranulation indicator fusion protein are provided. The nucleic acid molecules may be contained with a vector such as an expression vector. The instant invention also encompasses recombinant cells, particularly NK cells, expressing at least one degranulation indicator fusion protein.

In accordance with another aspect of the instant invention, methods for identifying an agent which modulates the degranulation process are provided. In a particular embodiment, the method comprises contacting a cell expressing at least one degranulation indicator fusion protein with at least one agent; and monitoring the signal from the pH sensitive detectable agent of the degranulation indicator fusion protein, wherein a change in the signal from the pH sensitive detectable agent in cells contacted with the agent compared to the signal from cells which were not contacted with the agent indicates that the agent is a modulator the degranulation process. In a particular embodiment, the method further comprises activating the cells prior to, simultaneously, and/or after contact with the agent.

### BRIEF DESCRIPTION OF THE DRAWINGS

FIG. 1 shows the kinetics and sustenance of actin accumulation at the activated IS. FIG. 1A: GFP-actin expressing NK-92 cells were activated on immobilized antibody to NKp30 and CD18 and imaged by TIRF microscopy. Images were acquired over 50 minutes at a rate of 1 frame per minute. Images of a representative cell are shown at 5-minute intervals beginning following 2.5 minutes of contact. Scale bar=5 μm. FIG. 1B: Area and mean fluorescence intensity (MFI) for 6 cells plotted over time (error bars, ±SD). Data are representative of three independent experiments.

FIG. 2 shows a timecourse of degranulation of activated NK-92 cells. NK-92 cells were activated by immobilized antibody to NKp30 and CD18 and incubated at 37° C. Supernatants were harvested at indicated times and assayed for Granzyme A activity using the BLT Esterase assay and results are shown as a percent of total potential release. Single antibody control supernatants were harvested following 60 minutes of activation. Values shown represent the mean+SD of 3 independent experiments.

FIG. 3 shows that granule approach to the synaptic membrane is coincident with the actin network. FIG. 3A: NK-92 cells expressing GFP-actin were loaded with LysoTracker® Red and activated by immobilized antibody to NKp30 and CD18. Cells were imaged at 1 frame per minute for 60 minutes using TIRFm. Images are shown at 5 minute intervals starting at 10 minutes following contact. Scale bar=5 μm. FIG. 3B: The intensity of GFP-actin fluorescence at the point of granule approximation was determined by calculating the ratio of the MFI of GFP-actin in the granule region to the MFI of the whole cell footprint. FIG. 3C: Minimum (min) and maximum (max) possible values are plotted along with the mean. Min and Max values were determined by using the minimum and maximum pixel values of the actin footprint and the equation described in FIG. 3B. FIG. 3D: GFP ratio values for 14 cells plotted as in FIG. 3A.

FIG. 4 shows a model and implementation of pHluorin-LAMP1 construct. FIG. 4A: Model of the construct depicting relative locations of sequences: endoplasmic reticulum targeting signal sequence (SS), flexible glycine-serine linker (GS), transmembrane domain (TM). FIG. 4B: Diagram depicting fluorescent state of pHluorin depending on intralumenal versus surface location. FIG. 4C: Sequence information for pHlourin-LAMP1 construct (SEQ ID NO: 1). FIG. 4D: Amino acid sequence of pHlourin-LAMP1 construct (SEQ ID NO: 2).

FIG. 5 shows pHluorin-LAMP1 reports degranulation in locally hypodense regions of actin. FIG. 5A: Histogram demonstrating green fluorescence measured by flow cytometry of NK-92 cells expressing pHluorin-LAMP1. Cells were untreated or treated with PMA/Ionomycin or CMA. FIG. 5B: NK-92 cells expressing pHluorin-LAMP1 were loaded with LysoTracker® Red and imaged by TIRF under activating conditions. Frames were acquired at a rate of 2 frames per min following 10 minutes of activation. FIG. 5C: NK-92 cells expressing pHluorin-LAMP1 and mCherry-actin were activated by immobilized antibody to NKp30 and CD18 and imaged by TIRFm. Images shown were taken at 10-second intervals at indicated time of activation. Scale bar=5 μm. FIG. 5D: Ratio measurements of the MFI of mCherry-actin at the site of degranulation to the MFI of the whole cell footprint were calculated and represent 52 events; mean=0.965. FIG. 5E: Image from FIG. 5A overlaid with concentric circles starting from the centroid of a degranulation event demonstrate measurement strategy for regional actin fluorescence intensity. FIG. 6F: Radial intensity profiles of pHluorin and mCherry MFI are depicted and show the signal intensity changes as circles are moved radially outward from the centroid of degranulation event (left to right). FIG. 5F: Radial intensity change of sequential circles moving outward from the centroid. Data represent 52 degranulation events (error bars, ±SD). Values are statistically significant (**, p<0.01, one sample t-test) compared to a value of 0.0, which would represent no change between sequential circles.

FIG. 6 shows degranulation events are less abundant than granule approximations. FIG. 6A: pHluorin-LAMP1 expressing cells were loaded with LysoTracker® Red and imaged for approximately 60 minutes at a rate of 1 frame per minute. FIG. 6B: To count events, all frames from the acquisition were merged into a single image. FIG. 6C: Number of LysoTracker® positive and pHluorin positive events for each cell are plotted (N=27; ***, p<0.0001, paired t-test).

FIG. 7 shows inhibiting actin dynamics after activation interferes with degranulation. NK-92 cells were activated by immobilized antibody to NKp30 and CD18 and incubated at 37° C. Indicated inhibitors were added to samples at 0 minutes, 10 minutes, or 20 minutes following activation. Supernatants were harvested after 60 minutes of total incubation and assayed for Granzyme A activity. All values are statistically significantly different from respective DMSO controls (range: p<0.001-0.05, unpaired t-test) and represent the mean of three experiments (error bars, =SD).

FIG. 8 shows lytic granule polarization precedes persistence at the NK immunological synapse prior to target cell death. YTS GFP-actin (FIGS. 8A, 8B, 8C) or NK92 GFP-tubulin (FIGS. 8D, 8E) NK cells were loaded with LysoTracker® Red and incubated with CellMask® labeled 721.221 (FIGS. 8A, 8B, 8C) or K562 (FIGS. 8D, 8E) target cells in the presence of SYTOX® Blue to detect cell death. Conjugates were imaged by confocal microscopy at 1 frame per minute for 90-130 minutes. FIG. 8A: Representative YTS GFP-actin-721.221 conjugate is shown at 10-minute intervals for 120 minutes. Scale bar=1 μm. FIG. 8B: Time to granule polarization in YTS GFP-actin NK cells as measured by lytic granule centroid distance to IS (mean 41.5±12 minutes, n=10). FIG. 8C: Time to initiation of 721.221 target cell apoptosis as detected by SYTOX® Blue entry is shown for 10 conjugates (mean 61.5±10 minutes, n=10). FIG. 8D: Time to granule polarization in NK92 GFP-tubulin NK cells as measured by lytic granule centroid distance to IS (mean 24.2 minutes, n=6). FIG. 8E: Time to initiation of K562 target cell apoptosis as detected by SYTOX® Blue entry is shown for 10 conjugates (mean 54.2 minutes, n=6). All representative images and analyses shown are from 4 independent experiments.

FIG. 9 shows lytic granules navigate the cell cortex prior to degranulation. NK92 cells expressing pHlourin-LAMP1 were loaded with LysoTracker® Red and activated by immobilized antibody to NKp30 and CD18. Cells were imaged by TIRFm at 6 frames per minute for 60-80 minutes. FIG. 9A: Representative NK92 lytic granule is shown at 5-minute intervals following 10 minutes of cell contact with the activating surface. Granules were tracked using Volocity® software prior to and following degranulation as described in Materials and Methods. Pre-degranulation LysoTracker® Red and post-degranulation pHluorin-LAMP1 tracks are shown in the final 55-minute image. Scale bar=1 μm. FIG. 9B: Overlay of LysoTracker® Red tracks of 14 pre-degranulation events over 4 separate experiments. Lytic granule track from FIG. 9A shown in bold. FIG. 9C: Overlay of pHluorin-LAMP1 tracks of corresponding degranulation events. Lytic granule track from FIG. 9A shown in bold.

FIG. 10 shows synaptic lytic granules show greater motility prior to degranulation. Granule tracks were analyzed using Volocity® software. Granule track length (FIG. 10A), track velocity (FIG. 10B), displacement (FIG. 10C), and displacement rate (FIG. 10D) from 14 events pre- (LysoTracker® Red, left) and post-degranulation (phlourin-LAMP1, right) are shown. Representative NK92 lytic granules from FIG. 9A are indicated by open diamonds. Mean±SD are shown. Differences between LysoTracker® Red and pHlourin-LAMP1 granule tracks were significant (p<0.0001, two-tailed t test). Results are from 4 independent experiments.

FIG. 11 shows lytic granules that do not degranulate show normal synaptic motility. Lytic granules demonstrating no observed degranulation were analyzed. FIG. 11A: Representative NK92 lytic granule cropped from image sequence is shown at 5-minute intervals following 10 minutes of contact. LysoTracker® Red track denoting all observed timepoints is shown in final 55-minute image. Scale bar=1 μm. FIG. 11B: Overlay of LysoTracker Red tracks of 10 lytic granules over 4 separate experiments. Representative granule track from FIG. 11A is depicted in bold.

FIG. 12 shows characteristics of synaptic lytic granule motility that do not degranulate. Comparative measurements of LysoTracker® Red motility in lytic granules for which degranulation or no degranulation was observed. Length (FIG. 12A), track velocity (FIG. 12B), displacement (FIG. 12C), displacement rate (FIG. 12D), and persistence time (FIG. 12E) plotted for 10 granules observing no degranulation (LysoTracker® Red only). Data is shown alongside measurements obtained from degranulation events previously illustrated in FIG. 10 (i.e., the same results in FIG. 10 for degranulating granules). The representative NK92 granule from FIG. 11A is indicated in LysoTracker® Red data set for comparison to the other granules measured, but for which image sequences are not shown (open diamond). Mean±SD are depicted. The means of the LysoTracker® Red data sets were not significantly different (p>0.05, two-tailed t test). Results are shown from 4 independent experiments.

FIG. 13 shows the effect of actin depolymerization upon synaptic lytic granule kinetics. NK92 cells expressing pHlourin-LAMP1 were loaded with LysoTracker® Red and activated upon immobilized antibody to NKp30 and CD18. Cells were imaged by TIRF microscopy at 6 frames per minute for 60-80 minutes. A representative NK92 lytic granule cropped from the image sequence is shown at 5-minute intervals following 5-10 minutes of contact-induced activation. LysoTracker® Red and pHluorin-LAMP1 tracks depicting the course of the granule over all timepoints are shown in final 55-minute image. Scale bars=1 μm. FIG. 13A: Vehicle control (DMSO) was added 10-20 minutes following the addition of cells to the imaging chamber. The white circle indicates granules location in frames 1-5. FIG. 13B: LatA was added between 10 and 15 minutes for a final concentration of 10 μM. FIG. 13C: Measured mean characteristics of synaptic lytic granule motility before (black bars) and after (white bars) degranulation. Mean track length, track velocity, displacement, displacement rate, and timespan of lytic granules are all shown relative to the respective DMSO values, which have been normalized to 1. Mean±SD are shown. Significant differences between DMSO- and Latrunculin A-treated granule tracks are marked with an asterisk (p<0.05, two-tailed t test). Results shown are from 4 independent experiments.

FIG. 14 shows the synaptic actin network is required for the persistence of degranulation. FIG. 14A: The lifetime of lytic granules post-degranulation in NK cells treated with LatA or DMSO control. Time points reflect amount of time elapsed post-degranulation (marked by the appearance of LAMP1-pHluorin) with vertical drops indicating disappearance of the granule from TIRF field. Vertical ticks indicate granules persisting to the end of the imaging sequence. FIG. 14C: Sum fluorescent intensity of lytic granules in NK92 cells expressing pHluorin-LAMP1. Cells were treated with DMSO (solid), or LatA (dashed) as per FIG. 13. Note that sum fluorescent intensity is a function of both the area and mean fluorescent intensity of a lytic granule. FIG. 14C: Area of the observed lytic granules in cells treated with DMSO (solid) or LatA (dashed). Granule boundaries were defined using fluorescent intensity with 3 SD above background as a cutoff. Results shown are from 4 independent experiments.

FIG. 15 shows the effect of actin depolymerization upon degranulating ganule area and LAMP1-pHluorin intensity. Analyses depict the first 20 minutes post-degranulation to demonstrate mean data from multiple events. FIG. 15A: Mean sum fluorescent intensity of lytic granules in NK92 cells expressing pHluorin-LAMP1. Cells were treated with DMSO (solid) and Latrunculin A (dashed). FIG. 15B: Size of the observed lytic granules in cells treated with DMSO (solid) and LatA (dashed). Time points reflect elapsed time post-degranulation. Differences in sum fluorescent intensity between the two groups are statistically significant by the Mann-Whitney U test (p>0.05) whereas there is no significant difference in area. Results shown are from four independent experiments, n=12 (control), n=18 (LatA).

FIG. 16 shows a model for the role of actin in granule persistence. A LysoTracker® Red-loaded granule is depicted approaching within cell cortex nearing a region within the F-actin network suitable for membrane access (1). As docking and fusion occurs (2), F-actin acts as a tether to help anchor the granule at the membrane, although in both cases fusion results in the activation of LAMP1-pHluorin and the subsequent appearance of green fluorescence. In addition, actin reorganization is likely to act in the generation of force to aid in the focused expulsion of granule contents (as supported by greater area*intensity of pHluorin-LAMP1 in control-compared to LatA-treated cells) (3) and the continued persistence of the degranulating granule at the cortex which may be a feature of the interaction of the granule with the local F-actin network (4).

### DETAILED DESCRIPTION OF THE INVENTION

The present invention provides reagents and methods which are useful for identification and biochemical characterization of agents which modulate the degranulation process in T lymphocytes. Natural killer and T cells kill target cells through secretion of lysosomal contents, a process termed degranulation. A pH sensitive, fluorescent fusion protein, termed pHluorin-LAMP1 has been engineered for use in microscopy or other fluorescence based applications for accurate and specific detection of degranulation events in real time.

pHluorin-LAMP1 is a chimeric fusion protein which includes the necessary sequence information directing transit of the expressed protein to the luminal side of the lysosomal membrane. It also includes the sequence for ecliptic pHluorin, a mutant form of GFP which does not fluoresce at acidic pH (below 6) but does fluoresce at neutral pH. Because of this pH sensitivity, pHluorin will not fluoresce if localized in the lysosome where the pH is about 5.5. pHluorin-LAMP1 comprises the endoplasmic reticulum directing signal sequence from IL2-Ra operably linked to ecliptic-pHluorin which is in turn linked via a linker of repetitive glycine-serine residues to the transmembrane domain of IL-2Ra which is linked to the cytoplasmic tail of lysosomal-associated membrane protein 1 (LAMP1) which is the segment which directs sorting of the fusion protein to the lysosome. Additionally, LAMP1 is detectable on the cell surface upon degranulation. As the cytoplasmic tail is the only portion required for appropriate LAMP1 trafficking, the pHluorin molecule will traffic as LAMP1 does, and thus will be exposed on the cell surface following activation induced degranulation.

The pH sensitive fusion protein described above has utility in degranulation studies because it is a fluorescent fusion protein that is essentially invisible until a cell is activated and degranulates. Below, successful transduction of NK cells with a construct encoding pHluorin-LAMP1 is shown. It is demonstrated that when these cells are in a resting state, the protein cannot be detected. When the cell is activated to degranulate, pHluorin-LAMP1 fluoresces at the cell surface, an event that is detectable via fluorescent microscopy. Thus, pHluorin-LAMP1 can be used in degranulation assays in order to identify agents which interfere with or augment this process.

The microscopy techniques and reagents provided herein exhibit enhanced sensitivity and resolution over those used previously and can be used to advantage to investigate the NK cell immunologic synapse (IS) as described further below. It is shown that F-actin is present throughout the synapse and that lytic granules likely navigate and are secreted through the filamentous network by accessing minimally sufficiently sized clearances. These data demonstrate a previously unappreciated distribution of F-actin at the NK cell IS and define an unrecognized mode of granule access to the synaptic membrane and functional secretion in lymphocytes.

Degranulation Indicator

As stated hereinabove, the instant invention encompasses degranulation indicators. In a particular embodiment, the degranulation indicator is a fusion protein comprising a pH sensitive detectable agent operably linked to a lysosome targeting moiety (e.g., LAMP1 or a fragment thereof). In a particular embodiment, the amino acid sequence of the degranulation indicator has at least 80%, 85%, 90%, 95%, 97%, 99%, or 100% identity with SEQ ID NO: 2.

The pH sensitive detectable agent may be detectable at acidic pH (e.g., about pH 6 or lower) and undetectable at neutral and/or basic pH or may be undetectable at acidic pH (e.g., about pH 6 or lower) and detectable at neutral and/or basic pH. Typically, the pH sensitive detectable agent is undetectable at acidic pH and detectable at neutral and/or basic pH. The detectable agent may be any compound or protein which may be assayed for directly or indirectly, particularly directly. Detectable agents include, for example, chemiluminescent, bioluminescent, and fluorescent compounds or proteins. In a particular embodiment, the detectable agent is fluorescent protein. Fluorescent proteins include, without limitation, green fluorescent protein (GFP), red fluorescent protein (RFP), and their derivatives. In a particular embodiment, the pH sensitive detectable agent is pHluorin (see, e.g., Miesenböck et al. (1998) Nature 394:192-5), mNectarine (Johnson et al. (2009) J. Bio. Chem., 284:20499-20511), or those described in Bizzarri et al. (Anal. Bioanal. Chem., 393:1107-22). The pHluorin may have an amino acid sequence having at least 80%, 85%, 90%, 95%, 97%, 99%, or 100% identity with SEQ ID NO: 3.

As stated hereinabove, the pH sensitive detectable agent of the degranulation indicator is operably linked to a lysosome targeting agent. Examples of lysosome targeting agents include, without limitation, mannose-6-phosphate receptor, LAMP1, LAMP2, lysosome integral membrane protein-1 (LIMP-1), LIMP-2, sortilin, or fragments thereof (e.g., the cytoplasmic tail or lysosome targeting domain) (Ogata et al. (J. Biol. Chem. (1994) 269:5210-5217; Ni et al. (Histol. Histopathol. (2006) 21:899-913). In a particular embodiment, the pH sensitive detectable agent of the degranulation indicator is operably linked to the LAMP1 cytoplasmic tail. The LAMP1 cytoplasmic tail may have an amino acid sequence having at least 80%, 85%, 90%, 95%, 97%, 99%, or 100% identity with SEQ ID NO: 4. The LAMP1 cytoplasmic tail may be longer or shorter than SEQ ID NO: 4 by about 1, 2, 3, 4, or 5, amino acids, particularly 1 or 2 amino acids, at the N-terminus and/or C-terminus (particularly N-terminus).

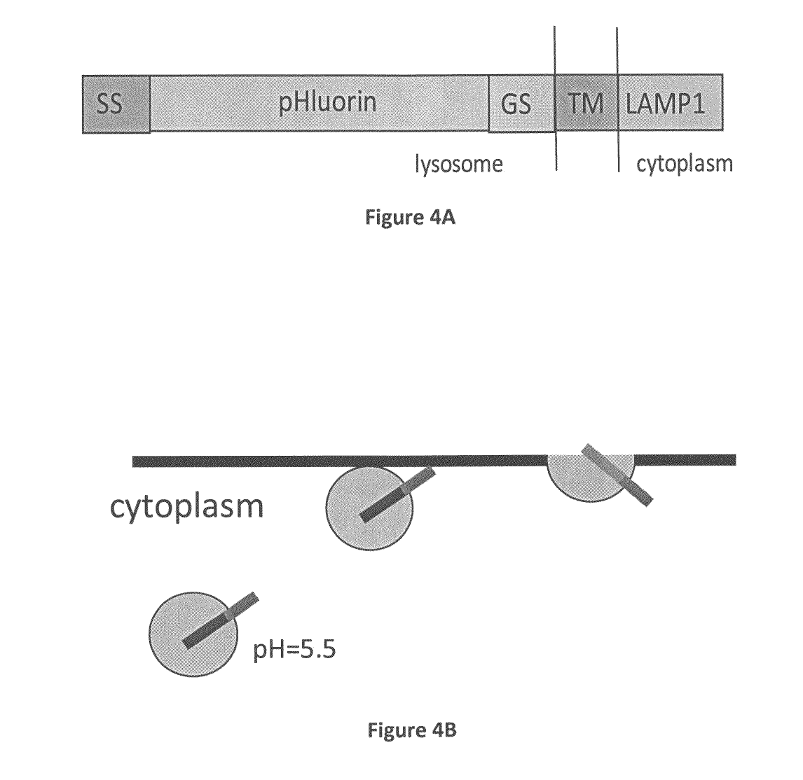

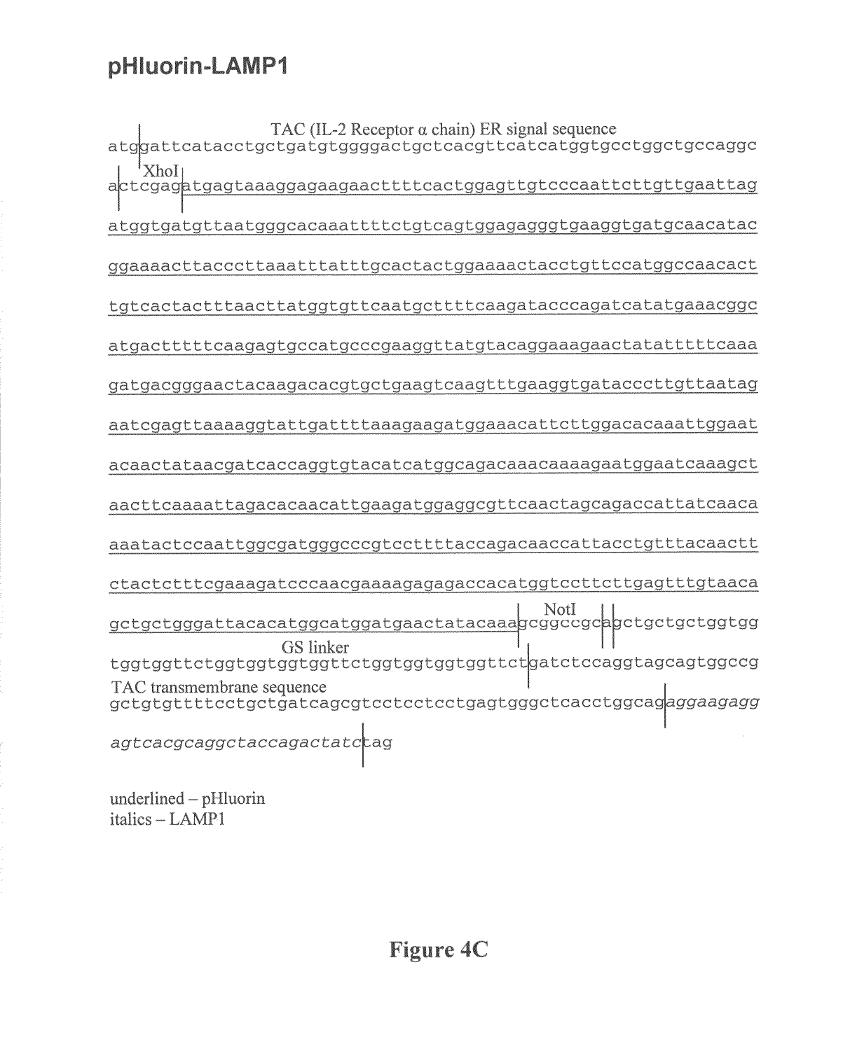

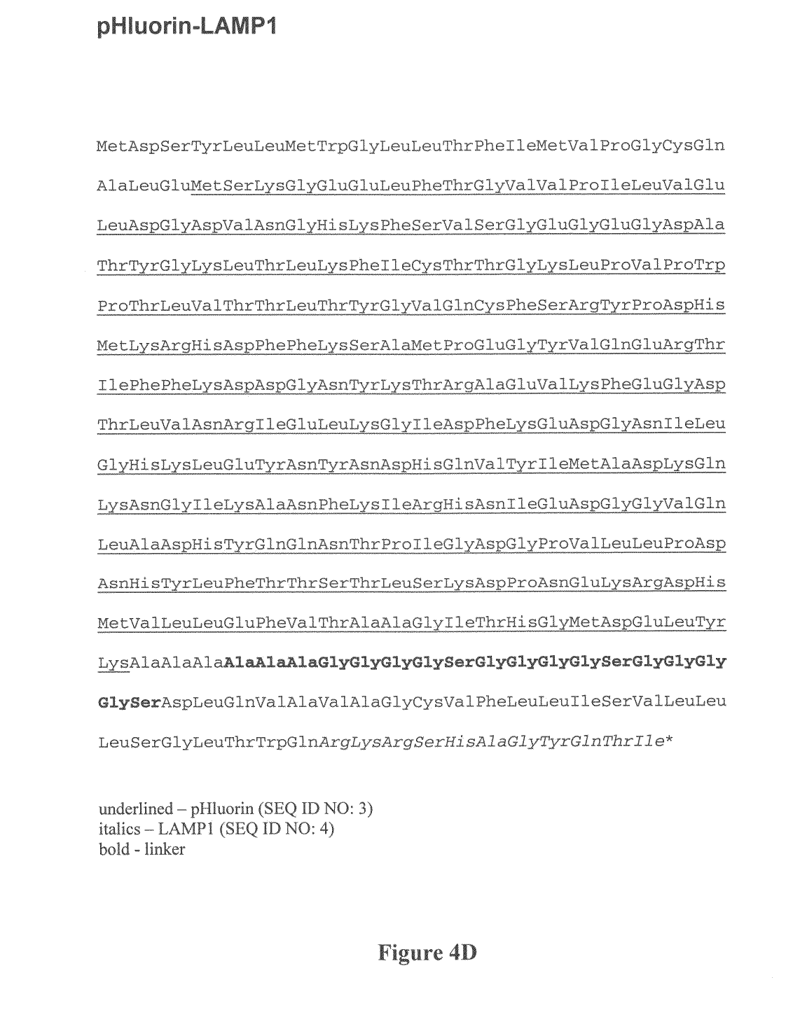

The degranulation indicator may further comprise a transmembrane domain. The transmembrane domain may be immediately adjacent (e.g., N-terminal) to the lysosome targeting agent or may be operably linked via a linker (e.g., 1 to about 10 amino acids, particularly 1 to about 5 amino acids). A transmembrane domain is the region of a transmembrane protein that spans across the lipid bilayer membrane of a cell. Typically, a transmembrane domain will be an amino acid sequence which is at least about 15 to 35, particularly about 20 to 30, amino acid residues in length and which contains at least about 65-70% hydrophobic amino acid residues (e.g., alanine, leucine, phenylalanine, protein, tyrosine, tryptophan, or valine). In a particular embodiment, the transmembrane domain is the transmembrane domain of the IL-2 receptor α chain. The transmembrane domain of the IL-2 receptor α chain may have an amino acid sequence having at least 80%, 85%, 90%, 95%, 97%, 99%, or 100% identity with the transmembrane sequence provided in FIG. 4D.

As depicted in FIG. 4A, the degranulation indicator may further comprise a linker domain operably linking the pH sensitive detectable agent and the transmembrane domain. In a particular embodiment, the linker comprises 1 to about 25, 1 to about 20, 1 to about 15, or 1 to about 10 amino acids. The linker may be a flexible linker such as a (G₄S)ₓ linker, where x is typically about 2 to about 5. The linker may have an amino acid sequence having at least 80%, 85%, 90%, 95%, 97%, 99%, or 100% identity with the linker sequence provided in FIG. 4D.

The degranulation indicator may further comprise an endoplasmic reticulum targeting signal sequence. The endoplasmic reticulum targeting signal sequence may be immediately adjacent (e.g., N-terminal) to the pH sensitive detectable agent or may be operably linked via a linker (e.g., 1 to about 10 amino acids, particularly 1 to about 5 amino acids). The endoplasmic reticulum targeting signal sequence can be from any protein (see, e.g., www.signalpeptide.de/). In a particular embodiment, the endoplasmic reticulum targeting signal sequence is the endoplasmic reticulum targeting signal sequence of the IL-2 receptor α chain. The endoplasmic reticulum targeting signal sequence of the IL-2 receptor α chain may have an amino acid sequence having at least 80%, 85%, 90%, 95%, 97%, 99%, or 100% identity with the endoplasmic reticulum targeting signal sequence provided in FIG. 4D.

The instant invention also encompasses nucleic acids encoding the degranulation indicator of the instant invention. Nucleic acids of the present invention may be maintained in any convenient vector (e.g., plasmid) or viral vector, particularly an expression vector. Different promoters may be utilized to drive expression of the nucleic acid sequences based on the cell in which it is to be expressed. Antibiotic resistance markers are also included in these vectors to enable selection of transformed cells. Protein encoding nucleic acid molecules of the invention include cDNA, genomic DNA, DNA, RNA, and fragments thereof which may be single- or double-stranded. The instant invention also encompasses recombinant cells (e.g., lymphocyte cells, particularly NK cells) comprising nucleic acids encoding the degranulation indicator of the instant invention (e.g., in a vector).

Compositions comprising at least one degranulation indicator; at least one nucleic acid encoding a degranulation indicator; or at least one recombinant cell of the instant invention and at least one carrier are also encompassed by the instant invention. Kits comprising at least one degranulation indicator; at least one nucleic acid encoding a degranulation indicator; at least one recombinant cell of the instant invention; and/or at least one of the above compositions are also encompassed by the instant invention. The kits may also comprise at least one NK activator.

As described in Example II, the instant invention also encompasses screening methods to identify modulators of the degranulation process. In a particular embodiment, the method comprises contacting a cell comprising the degranulation indicator of the instant invention (e.g., a cell expressing the degranulation indicator from a vector encoding the same) with at least one agent and monitoring (e.g., measuring the strength, monitoring the location, measuring the duration, etc.) the signal/activity from the pH sensitive detectable agent (e.g., the fluorescence)), wherein a change the signal/activity from the pH sensitive detectable agent indicates that the tested agent modulates the degranulation process. In a particular embodiment, the signal/activity from the pH sensitive detectable agent is measured over a period of time. The agent may modify/modulate any part of the degranulation process. The agents may, without limitation, modulate activation of the degranulation process, actin accumulation at relevant sites, granule transport/secretion, and/or membrane localization.

The agent tested by the methods of the instant invention can be any compound (e.g., an isolated compound), particularly any drug (e.g., an FDA approved drug), organic molecule, or small molecule. For example, the compound may be a polypeptide, protein, peptide, nucleic acid molecule (e.g., encoding a protein of interest), inhibitory nucleic acid molecule (e.g., antisense or siRNA), organic compound, inorganic compound (e.g., heavy metals, mercury, mercury containing compounds), or small molecule. In a particular embodiment, a library of agents is screened.

### Definitions

As used herein, a linker is a chemical moiety comprising a covalent bond or a chain of atoms that covalently attaches two molecules to each other. In a particular embodiment, the linker contains amino acids, particularly from 1 to about 50, 1 to about 25, 1 to about 20, 1 to about 15, 1 to about 10, or 1 to about 5 amino acids.

The term “vector” refers to a carrier nucleic acid molecule (e.g., DNA) into which a nucleic acid sequence can be inserted for introduction into a host cell where it will be replicated. An “expression vector” is a specialized vector that contains a gene or nucleic acid sequence with the necessary regulatory regions needed for expression in a host cell.

As used herein, the term “small molecule” refers to a substance or compound that has a relatively low molecular weight (e.g., less than 4,000, particularly less than 2,000). Typically, small molecules are organic, but are not proteins, polypeptides, or nucleic acids, though they may be amino acids or dipeptides.

The term “isolated” may refer to a compound or complex that has been sufficiently separated from other compounds with which it would naturally be associated. “Isolated” is not meant to exclude artificial or synthetic mixtures with other compounds or materials, or the presence of impurities that do not interfere with fundamental activity or ensuing assays, and that may be present, for example, due to incomplete purification, or the addition of stabilizers.

A “carrier” refers to, for example, a diluent, adjuvant, excipient, auxilliary agent or vehicle with which an active agent of the present invention is contained. Carriers can be sterile liquids, such as water, aqueous saline solutions, and aqueous dextrose and glycerol solutions.

The following examples are provided to facilitate the practice of the present invention. They are not intended to limit the invention in any way.

### EXAMPLE I

### Degranulation Assay and Biochemical Characterization of the Immunologic Synapse (IS)

### Materials and Methods

Cell Lines and ex vivo NK Cells

NK-92 and GFP-actin expressing NK-92 cell lines (ATCC) were maintained in MyeloCult® (StemCell) media supplemented with 100 U/mL penicillin and streptomycin (Gibco) and 100 U/mL IL-2 (Hoffman-La Roche). mCherry-actin and pHluorin-LAMP1 expressing cells were generated by retroviral transduction of NK-92 cells as described (Sanborn et al. (2010) Methods Mol. Biol., 612:127-48). Briefly, 2-4 μg of plasmid DNA were transfected into the Phoenix packaging line using FuGENE® (Roche) lipofection reagent. Supernatant was harvested on day 2 post transfection. NK-92 cells, Polybrene® (Sigma), and supernatant were mixed and spun in a well of a 6 well plate at 1000×g for 90 minutes at 32° C. Following overnight incubation at 32° C., cells were spun down and resuspended in supplemented MyeoloCult®. Cells were grown for three days prior to the introduction of puromycin (2 μg/mL) (InvivoGen) or hygromycin B (150 μg/mL) (Cellgro). mCherry-actin expressing cells were sorted for high expression by the University of Pennsylvania Cell Sorting Facility.

Ex vivo NK cells were prepared from concentrated whole blood as described (Banerjee et al. (2007) J. Exp. Med., 204:2305-20).

### Generation of Plasmids

The pHluorin-LAMP1 retroviral plasmid was generated by BioMeans, Inc by inserting the sequence for pHluorin between the signal sequence and the transmembrane domain of IL-2Rα linked to the cytoplasmic tail of LAMP1. A flexible GS linker was added between pHluorin and the transmembrane domain sequences. The entire construct was subsequently cloned into the MIGR1-puromycin vector.

The mCherry-actin retroviral plasmid was generated by PCR amplifying mCherry-actin from a pmCherry plasmid with 5′ BglII and 3′ EcoRI restriction site overhangs. The PCR product was digested and ligated into the pMSCV-Hygromycin plasmid, which had an EcoRI site in the Hygromycin resistance gene sequence eliminated by site-directed mutagenesis.

### Flow Cytometry

Flow cytometry was performed to verify pHluorin-LAMP1 expression. Cells were untreated or treated with phorbol myristate acetate (PMA, 100 ng/mL,Sigma) and Ionomycin (1 μg/mL, Sigma) for 30 minutes or Concanamycin A (CMA, 100 nM, Sigma) for 90 minutes and samples were run on a Becton Dickinson FACSCalibur™.

### Total Internal Reflection Fluorescence Microscopy

Cells were washed and resuspended in supplemented Myelocult prior to use. For imaging of lytic granules, cells were incubated with 100 nM LysoTracker® Red DND-99 (Molecular Probes) for 30 minutes at 37° C., washed once and resuspended in supplemented Myelocult. ΔT dishes (Bioptechs) were coated with 5 μg/mL anti-NKp30 (Beckman-Coulter) and 5 μg/mL anti-CD18 (Clone IB4) for 1 hour at 37° C., washed with PBS and prewarmed prior to imaging with 1 mL dye free R10 (dye free RPMI 1640 (Gibco), 10% fetal bovine serum (Atlanta Biologicals), 10 mM HEPES (Gibco), 100 U/mL penicillin and streptomycin, 100 μM MEM nonessential amino acids (Gibco), 1 mM sodium pyruvate (CellGro), 2 mM L-glutamine (Gibco). 4×10⁵ cells were added to the dishes, which were maintained at 37° C. with a heated stage and lid (Bioptechs).

For fixed cell experiments, 1×10⁵ cells were adhered to 0.15 glass coverslips coated with antibody as described above. Samples were fixed and stained with Alexa Fluor 568 phalloidin (Molecular Probes) as described (Banerjee et al. (2007) J. Exp. Med., 204:2305-20).

Samples were imaged through a 1.49 NA, oil immersion, 60×, APO N TIRFm objective (Olympus) on an Olympus IX-81 with a rear mounted TIRF illuminator (Olympus). 488 nm (Spectra-Physics) and 561 nm (Cobalt) diode lasers were launched through a two-line combiner of an LMM5 (Spectral Applied Research). Lasers were aligned for total internal reflection prior to each experiment. Images were captured using Volocity (PerkinElmer) to control a C9100 EM-CCD camera (Hamamatsu).

### Confocal Micrsoscopy

Me11190 cells were plated into ΔT dishes one day prior to use. Cells were stained with CellMask™ Deep Red (Invitrogen) according to manufacturer's instructions just prior to imaging. GFP-actin expressing NK-92 cells were added to the dishes, which were maintained at 37° C., and imaged for up to one hour. Cells were imaged through a 63×1.4 NA Plan-APOCHROMAT objective (Zeiss) on a Zeiss Observer.Z1 using a C10600 ORCA-R2 camera (Hamamatsu). The microscope was equipped with a CSU10 spinning disc system (Yokogawa). 491 nm (Cobalt) and 655 nm (CrystaLaser) diode lasers were launched through an LMM5 (Spectral Applied Research).

### Degranulation Assay

Immulon 4HBX 96 well flat bottom plates (Thermo) were coated with murine IgG (BD), anti-human NKp30, anti-human CD18, or both anti-NKp30 and anti-CD18 at 5 μg/mL in PBS overnight at 4° C. Plates were washed twice with PBS and blocked for 1 hr at room temperature with R10 media. One×10⁵ cells were added to wells and plates were spun at 1000 rpm for two minutes before incubation at 37° C. For the timecourse degranulation assay, supernatants from spontaneous and activated wells were harvested at indicated times. For inhibitor treatments, media containing inhibitor or vehicle (DMSO) was added at indicated times. Inhibitors used were jasplakinolide (1 μM, Calbiochem), latrunculin A (10 μM, Sigma), cytochalasin D (10 μM, Sigma), and thapsigargin (1 μM, Calbiochem). Supernatatants were harvested after 60 minutes. For total release, cells were lysed in 0.5% Nonidet P40 (Accurate Chemical and Scientific). Supernatants were assayed by mixing 20 μL supernatant with 200 μL of a solution containing PBS, 9.8 mM HEPES (Gibco), 196 μM Z-L-Lys-SBz1 hydrochloride (BLT, Sigma), 218 μM 5,5′-dithiobis(2-nitrobenzoic acid) (DTNB, Sigma). Samples were incubated for 30 minutes at 37° C. and absorbance was measured immediately at 405 nm. Percent total release was measured by subtracting the spontaneous release value from activated release values (A-S) and the total release value (T-S), and then dividing (A-S) by (T-S).

### Image Analysis

Images and timelapse series were analyzed using either Volocity® or the FIJI package of ImageJ (pacific.mpi-cbg.de). Using Volocity®, fluorescently tagged actin footprints were identified using a classifier that identifies objects above a selected standard deviation above the mean intensity (usually 0-1). LysoTracker™ and pHluorin positive events were similarly identified (4-10 standard deviations above mean intensity), with the additional exclusion of events smaller than 0.05 μm². For actin fluorescence ratio measurements, the mean fluorescence intensity (MFI) at the location of granule approximation or degranulation was divided by the MFI of the actin footprint. Maximum and minimum actin intensities were also measured.

FIJI was used to generate radial intensity profiles using the radial profile plug-in (rsbweb.nih.gov/ij/plugins/radial-profile.html). For a profile of the entire cell footprint a region of interest (ROI) circle was drawn around the cell and data from running the plugin was exported to Excel (Microsoft). For profiles of degranulation events images were first split into red and green images and then a circle with a radius of 8 pixels (1.08 μm) was drawn around each event. Data was generated for 6 radii from the center of each event. To determine the radial intensity change, an outer radial intensity value was subtracted from an inner radial intensity value. Thus a negative value indicates that the outer value has higher mean intensity than the inner value.

To measure changes in the actin network over time, sequential images were imported into FIJI, a line was drawn across the center of the cell and line intensity profile data were generated for each time point at the same location within the cell. Data was exported to Excel and standard deviations calculated. Surface plots were generated using the surface plot function in FIJI.

Images from platinum replica electron microscopy were inverted and linearly contrast enhanced using Photoshop (Adobe) and imported into FIJI for processing and analysis. Background was subtracted from each image using the Rolling Ball Subtraction algorithm with the radius set to 25 pixels. Pixel intensities were subsequently squared to generate a measurable image. Cells were identified using the default autothreshold and cell contact area and cell centroid were measured with “include holes” checked. ROIs were drawn around the interior of the cell to more accurately identify filaments and to avoid debris. Filaments were identified using the default automatic threshold with “dark background” checked. Clearances in the filamentous network were identified by using the automatic threshold with “dark background” unchecked, and collecting only those objects with an area that would accommodate a granule with a diameter of 250 nm, 500 nm, or 750 nm. Accommodating area was determined by assuming that granules are spherical and thus determining the area of a circle that would accommodate them. More specifically, taking the radius of a 250 nm granule and using the equation for the area of a circle (A=πr²), the minimum size for a clearance is defined as 0.049 μm². Distance from the cell centroid was determined by inputing the coordinates of the cell centroid and a clearance into the Pythagorean equation (a²+b²=c²: (X_{centroid}−X_{clearance})²+(Y_{centroid}−Y_{clearance})²=c², where c is the distance from the cell centroid).

All data were plotted using Prism® (Graphpad).

### Statistical Analyses

Statistical significance was determined using Prism® to perform one-sample, unpaired, or paired, two-tailed Student's t-tests as indicated.

### Results

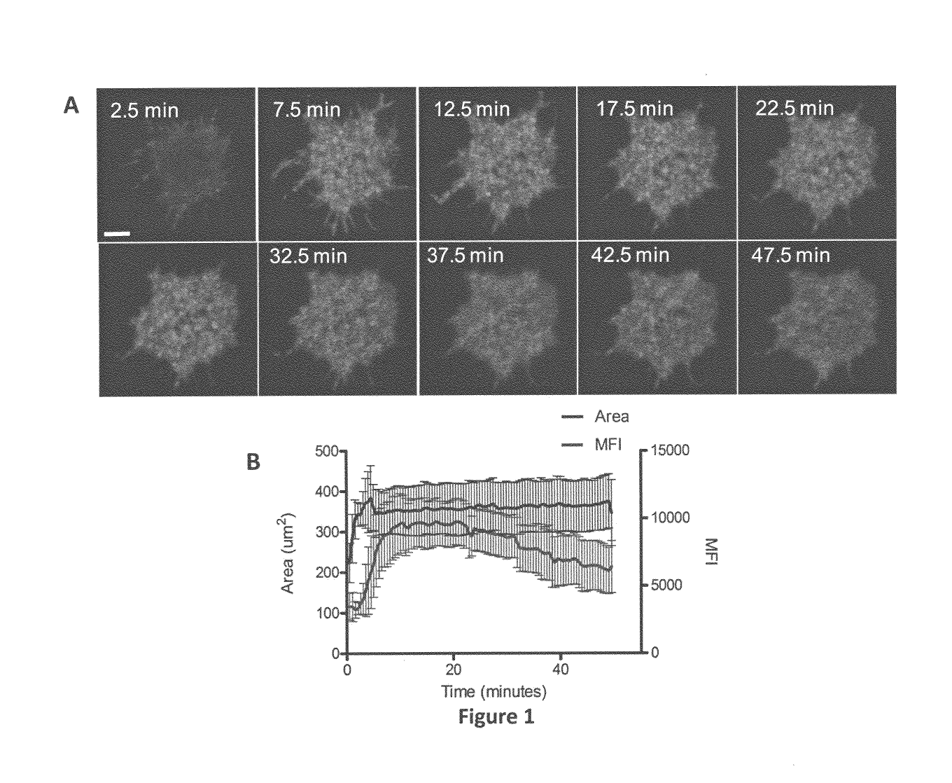

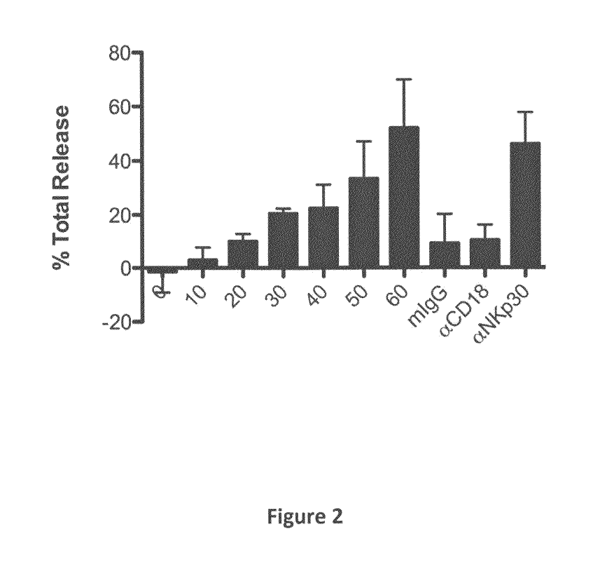

To evaluate the kinetics of actin accumulation at the activated synapse, NK-92 cells expressing GFP-actin were imaged using TIRFm after contacting an activating surface. Actin accumulated quickly within 5 minutes, and was sustained over the period of observation (50 minutes) (FIG. 1A). There was an initial paucity of actin at the synapse followed by a rapid filling in, as demonstrated by the separation of peak contact area and mean fluorescence intensity (MFI) of GFP-actin in that region (FIG. 1B). The decrease in MFI over time was due to photo-bleaching as separate imaging of fields at 10 and 40 minutes did not show MFI differences. Importantly, actin was diffusely accumulated prior to timepoints at which granule contents were detected in the supernatant (FIG. 2). Thus, actin was present as a potential barrier to lytic granule access to the plasma membrane.

### Lytic Granules Approximate the Synapse in Areas of Actin

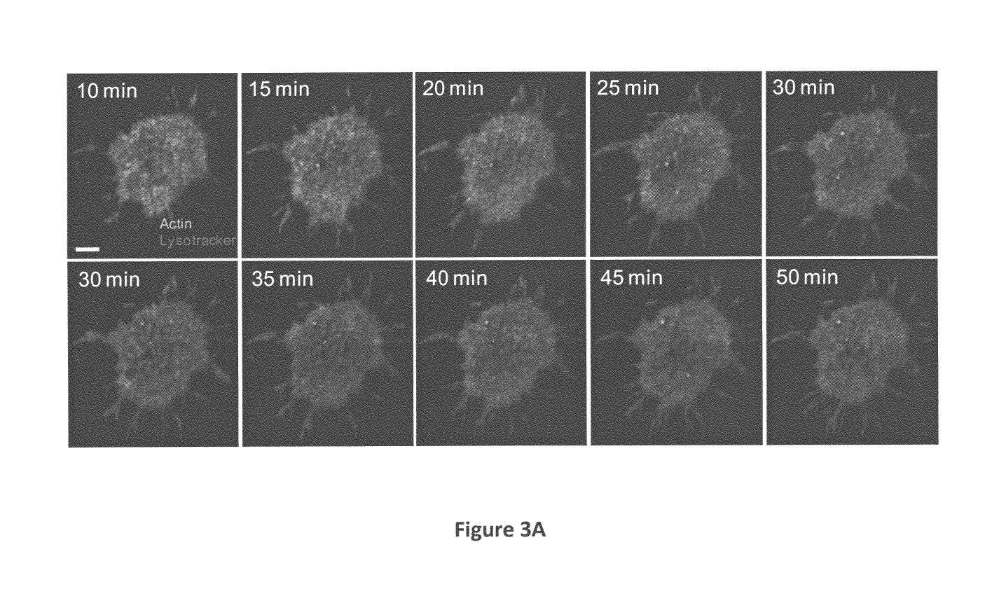

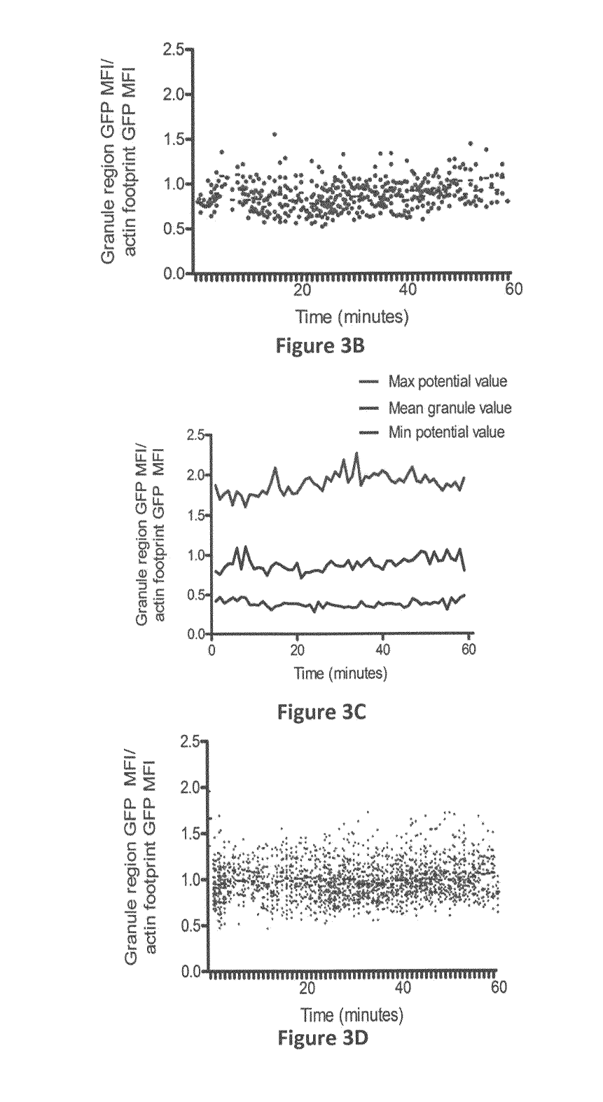

Because there was abundant actin present at the synapse, it was determined if lytic granules might utilize relative clearances in the actin network to access the synaptic membrane. To address this, GFP-actin expressing cells were loaded with LysoTracker® Red dye, which enables tracking of lytic granules and definition of their position relative to actin, and followed in real time after activation. Numerous granules were identified in the synaptic actin network using two-color TIRFm. Although some relative hypodensities were apparent in the synaptic actin, the LysoTracker® labeled granules did not necessarily appear in these relative voids of actin (FIG. 3A). To quantify this observation across all synaptic granules in an NK cell, the ratio of actin intensity in the region of the synaptic granule was compared to that of the entire synapse. The ratio of these two values demonstrated that on average granules approached the membrane in areas of at least some actin (FIG. 3B, 3C).

Combining measurements of all granules in the synapse over one hour from 14 cells defined the mean granule ratio value as 1.0 (FIG. 3D). Although there was a range of actin present throughout the synapse as measured by ratio of minimum and maximum intensity values to the MFI, few granules were present in areas of particularly low of high actin content. Thus, the colocalization of lytic granules with actin signal suggested that granules access the synapse in close proximity to the actin network.

There was, however, variability in colocalization between synaptic actin and granules (FIG. 3). Thus the possibility that an approximated granule might not necessarily be capable of degranulation was considered. Specifically, it was reasoned that granules which ultimately degranulate represent a subpopulation of approximation events. Furthermore, it was hypothesized that granules capable of degranulation might be those present within focal actin hypodensities. In order to study this directly, a novel degranulation indicator was developed for use in live cells.

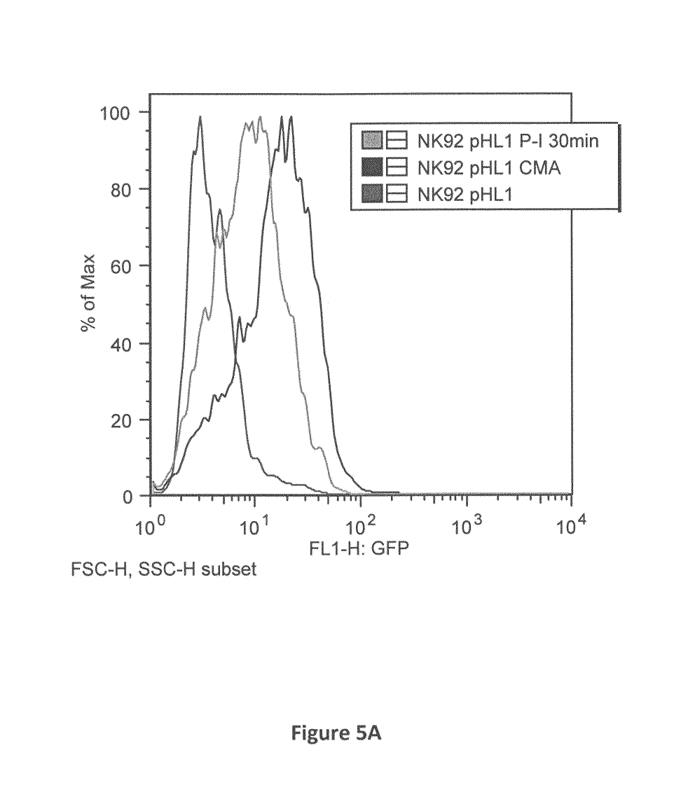

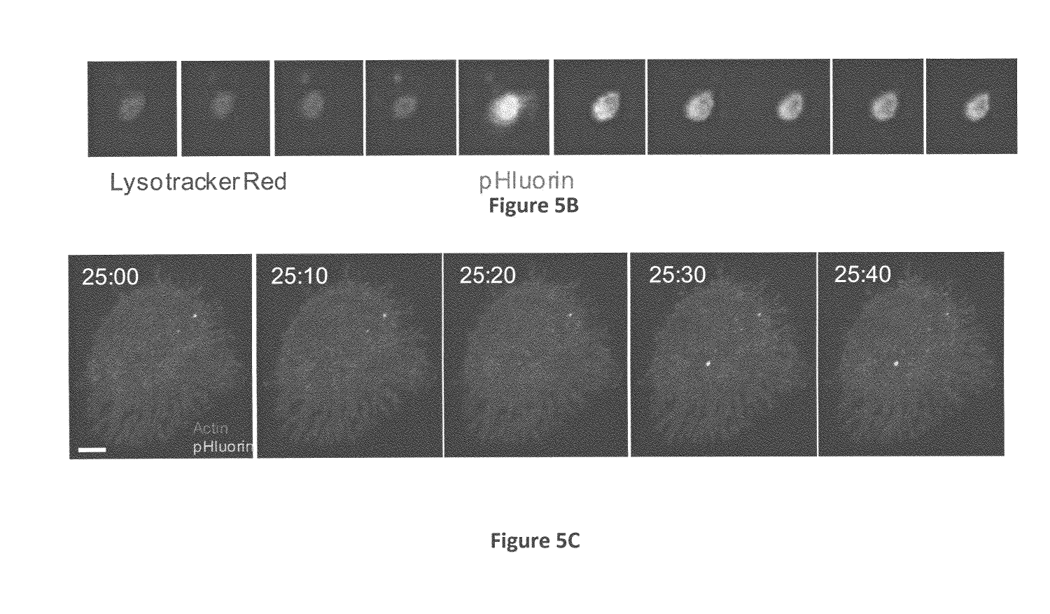

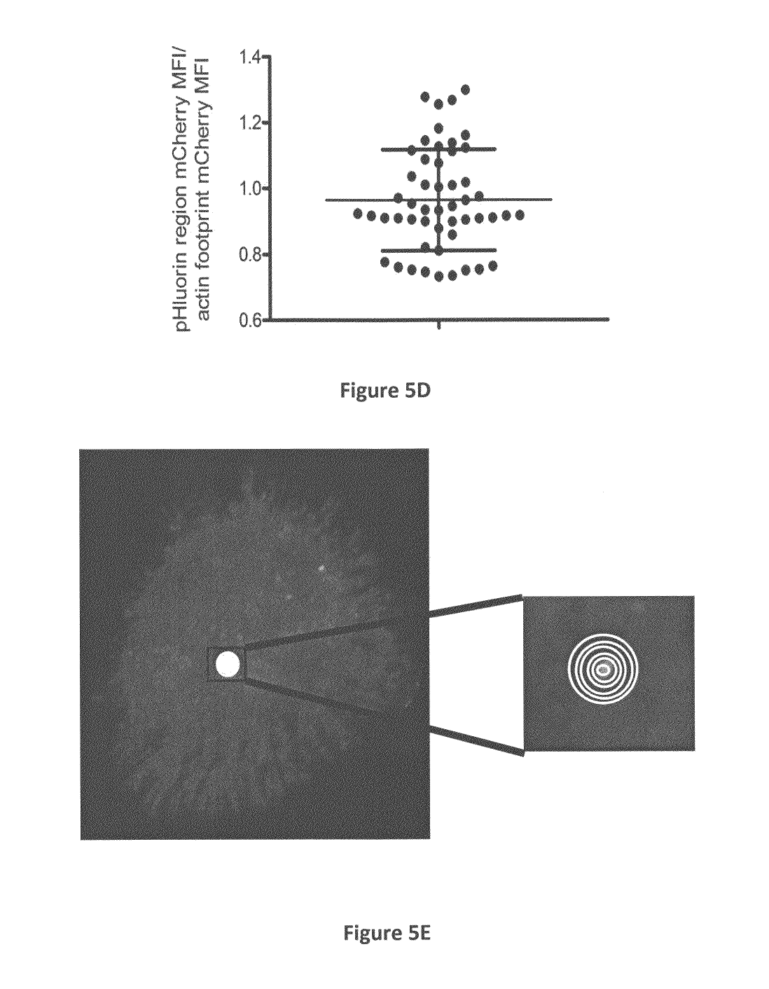

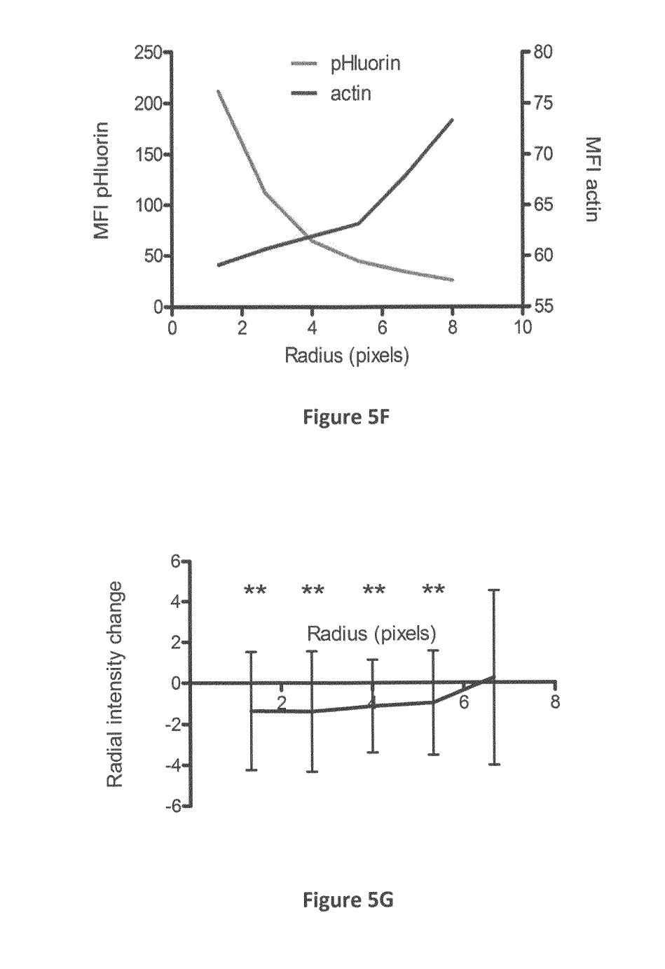

Lysosomal-associated membrane protein 1 (LAMP1, CD107a; e.g., GenBank Accession No. AAH93044), which is sorted to NK cell lytic granules (Peters et al. (1991) J. Exp. Med., 173:1099-109), is routinely used to detect cells that have degranulated by its appearance on the NK cell surface. Although previous investigations used antibody to LAMP1 to visualize degranulation (Liu et al. (2009) Immunity, 31:99-109), a cell-intrinsic approach was adopted by targeting a reporter fluorophore to the lytic granules. pHluorin, a pH sensitive mutant of GFP that does not fluoresce at acidic pH (Miesenbock et al. (1998) Nature, 394:192-5), was fused to the cytoplasmic tail of LAMP1 (FIG. 4A, 4B, 4C, 4D) and obtained stable expression in NK-92 cells. As expected with localization of the pHluorin-LAMP1 construct to lytic granules, treatment with concanamycin A (which effectively neutralizes lysosomal pH by inhibiting the vacuolar-type H+ ATPase) resulted in a robust increase in green fluorescence as measured by flow cytometry (FIG. 5A). Since degranulation is an activation-induced process, pHluorin-LAMP1 expressing cells were treated with the phorbol ester, PMA, and calcium ionophore, ionomycin, and found a rapid increase in pHluorin fluorescence, consistent with LAMP1 surface upregulation (FIG. 5A). To better define pHluorin-LAMP1 localization to acidic granules, LysoTracker® Red loaded pHluorin-LAMP1 expressing cells were studied using TIRFm after activation. Individual LysoTracker® Red labeled granules could be identified at the synapse and were observed to undergo a shift from red to green fluorescence (FIG. 5B). This event is consistent with the granule fusing with the synaptic membrane, releasing its contents, and encountering a pH neutral environment. These data are consistent with lytic granule targeting of pHluorin.

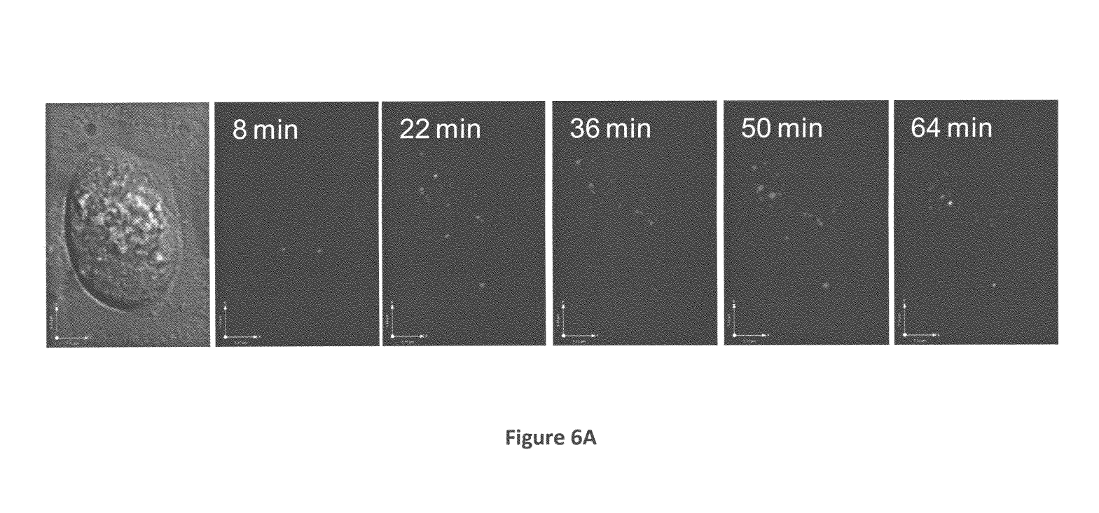

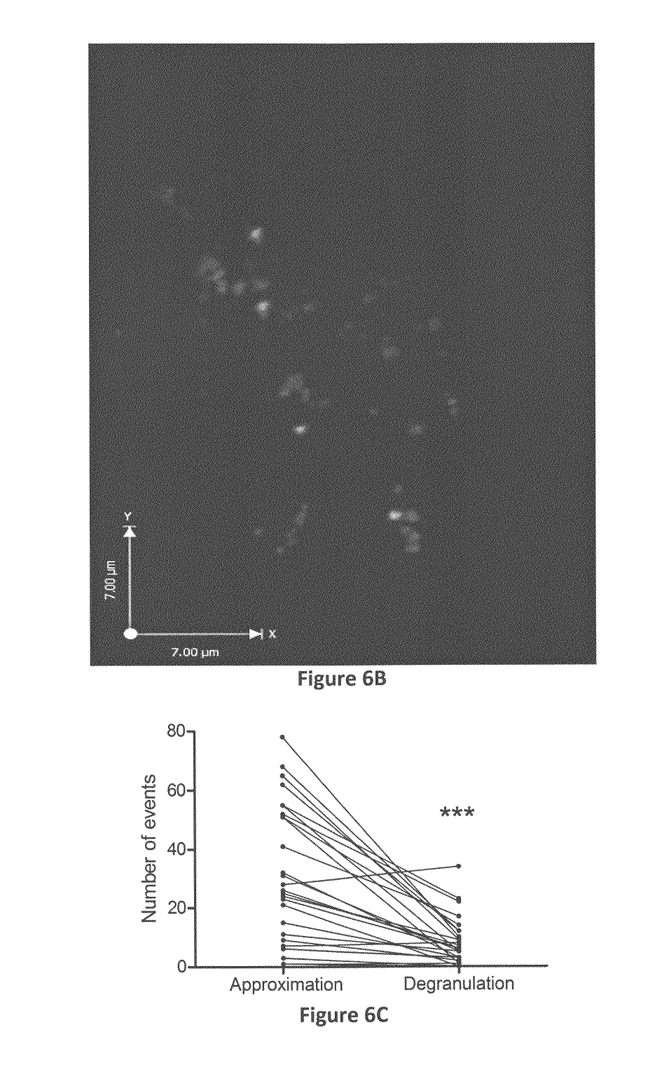

pHluorin-LAMP1 expressing NK-92 cells were then used to address whether granule approximation results in degranulation. LysoTracker® Red loaded, pHluorin-LAMP1 expressing cells were imaged over time using TIRFm (FIG. 6A). There were significantly more approximation events than degranulation events (mean=31 and 8 per cell, respectively) over one hour (FIG. 6B, 6C). Thus only a subset of granules that approximate the synaptic membrane result in a degranulation events.

Degranulation Occurs within Focal Actin Network Hypodensities

To directly investigate where degranulation occurs relative to the synaptic actin network, pHluorin-LAMP1 and mCherry-actin were stably coexpressed in cells and imaged them following activation using two-color TIRFm. Timelapse imaging demonstrated that degranulation events occurred in areas of at least some actin fluorescence, similar to that which was seen with granule approximations (FIG. 5C). The actin intensity at degranulation points was quantitatively evaluated by comparing the intensity values of the actin signal at the point of degranulation events to that of the entire cell contact. Degranulations were identified in regions of actin that had slightly lower signal than the mean actin signal of the cell footprint (ratio=0.965) (FIG. 5D). This indicated that degranulation events did not occur in areas of maximal actin but suggested that they occurred in close proximity to the actin network. The ratio for actin intensity at the site of degranulation events relative to that of the whole cell, however, was slightly lower than that calculated for approximation events (mean=0.965 and 1.0, respectively). This difference demonstrated that true degranulation events occurred in slightly hypodense areas of actin when compared to granule approximation events.

To further characterize the local actin network at the point of degranulation in consideration of focal hypodense regions, actin fluorescence was quantitatively evaluated in the immediate vicinity of degranulation events. Measurements of actin fluorescence were made along sequential pixel radii emanating from the centroid of individual degranulations extending approximately 1 μm outwards (FIG. 5E). The fluorescent intensities of actin as well as that of pHluorin were quantified in concentric circles along these radii (FIG. 5F). In general, as the pHluorin signal diminished from a degranulation centroid, the mCherry-actin signal increased suggesting that degranulation may occur in a focal actin hypodensity (FIG. 5F). To measure multiple events, the change in intensity between consecutive radiating circles was determined and plotted for all observed degranulation events (FIG. 5G). The mean value of the intensity change demonstrated reduced actin intensity at each of the innermost four radii compared to the neighboring outer radius, thus reflecting the example in FIG. 5F. This indicates that in moving from the periphery of the region of the degranulation event to its center the actin intensity decreased. Thus degranulation tends to occur in locally hypodense areas of the actin network (i.e. in regions with some but relatively less actin). In the presence of an actin barrier at the plasma membrane, therefore, hypodense regions of actin provide a potentially more accessible route to the synaptic membrane.

### Inhibiting Actin Dynamics After Activation Inhibits Degranulation

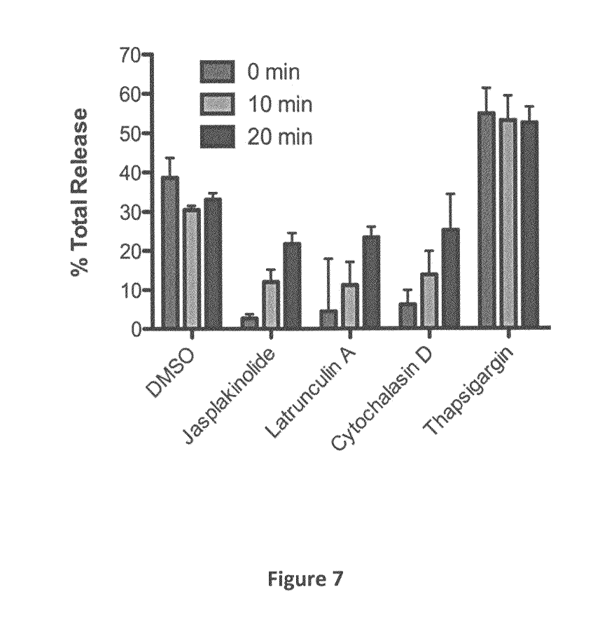

Since granules are in contact with at least some actin during degranulation, the role of actin dynamics in degranulation was investigated. Thus, actin dynamics was inhibited with drugs that prevent F-actin assembly (latrunculin A and cytochalasin D) or disassembly (jasplakinolide). Inhibitor addition at the time of activation almost completely inhibited degranulation (FIG. 7), a result that is consistent with the previously reported requirement for initial actin reorganization in synapse formation and maturation (Orange et al. (2003) Proc. Natl. Acad. Sci., 100:14151-6). In order to avoid inhibiting the initial, requisite actin reorganization (which was observed by 5 minutes (FIG. 1)), cells were treated with the inhibitors following 10 minutes of activation (a point at which minimal degranulation had occurred (FIG. 2)). Addition of the actin inhibitors after 10 minutes of activation resulted in an approximately 50% decrease in bulk degranulation (FIG. 7). Interestingly, addition of inhibitors at 20 minutes had only a marginal effect on degranulation. Thapsigargin, which has the net effect of elevating intracellular calcium levels, was used as a positive control and had a positive effect on degranulation. These results indicate that actin reorganization immediately prior to the start of degranulation, but after large-scale actin accumulation has occurred, is critical for subsequent granule release.

The work presented herein defines an actin network that is more pervasive at the NK cell IS than previously thought. Although this could serve as a potential barrier, abundant granule-sized clearances have been identified that could function as sufficient access points to the plasma membrane. These could provide functionality by allowing granules to pass between filaments and to simultaneously interact with them, whereby myosin IIA could exert force in squeezing granules between filaments or in post fusion expulsion of granule contents. This latter possibility has been suggested in chromaffin cells where myosin II function was required for appropriate release of catecholamines (Berberian et al. (2009) J. Neurosci., 29:863-70). Thus degranulation events at the NK cell IS represent a coupled interplay between actin filaments and clearances which presents additional opportunities to define regulatory steps important to NK cell cytotoxicity.

### EXAMPLE II

### Degranulation Assay and Reagents for Identification of Degranulation Modulating Agents

Example I provides a detailed description of a suitable degranulation assay and novel reagent for assessing degranulation events in real time. Also provided are exemplary chemical modulators of this process, such as jasplakinolide, latrunculin, cytochalasin D and thapsigargin. Various types of candidate drugs may be screened utilizing the compositions and methods described herein, including nucleic acids, polypeptides, small molecule compounds, and peptidomimetics. In some cases, genetic agents can be screened by contacting the engineered lymphocytes with a nucleic acid construct coding for a gene. For example, one may screen cDNA libraries expressing a variety of genes, to identify other genes that modulate the degranulation process. For example, the identified drugs may modulate activation of the degranulation process, actin accumulation at relevant sites, granule transport/secretion, and or, membrane localization. Accordingly, irrespective of the exact mechanism of action, drugs identified by the screening methods described herein are expected to provide therapeutic benefit in cases where lymphocyte activity must be enhanced or interfered with.

Screening methods described herein use may employ the recombinant cells described in Example I. Candidate drugs can be screened from large libraries of synthetic or natural compounds. One example is an FDA approved library of compounds that can be used by humans. In addition, compound libraries are commercially available from a number of companies including but not limited to Maybridge Chemical Co. (Trevillet, Cornwall, UK), Comgenex (Princeton, N.J.), Microsource (New Milford, Conn.), Aldrich (Milwaukee, Wis.), AKos Consulting and Solutions GmbH (Basel, Switzerland), Ambinter (Paris, France), Asinex (Moscow, Russia), Aurora (Graz, Austria), BioFocus DPI, Switzerland, Bionet (Camelford, UK), ChemBridge, (San Diego, Calif.), ChemDiv, (San Diego, Calif.), Chemical Block Lt, (Moscow, Russia), ChemStar (Moscow, Russia), Exclusive Chemistry, Ltd (Obninsk, Russia), Enamine (Kiev, Ukraine), Evotec (Hamburg, Germany), Indofme (Hillsborough, N.J.), Interbioscreen (Moscow, Russia), Interchim (Montlucon, France), Life Chemicals, Inc. (Orange, Conn.), Microchemistry Ltd. (Moscow, Russia), Otava, (Toronto, ON), PharmEx Ltd.(Moscow, Russia), Princeton Biomolecular (Monmouth Junction, N.J.), Scientific Exchange (Center Ossipee, N.H.), Specs (Delft, Netherlands), TimTec (Newark, Del.), Toronto Research Corp. (North York ON), UkrOrgSynthesis (Kiev, Ukraine), Vitas-M, (Moscow, Russia), Zelinsky Institute, (Moscow, Russia), and Bicoll (Shanghai, China). Combinatorial libraries are available and can be prepared. Libraries of natural compounds in the form of bacterial, fungal, plant and animal extracts are commercially available or can be readily prepared by methods well known in the art. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

For example, the recombinant cells in Example 1 can be incubated in the presence and absence of a test compound the effect of the compound on degranulation assessed. Agents so identified could then be tested in whole animal models to assess in vivo efficacy.

Agents identified using the screening assays described herein are also encompassed by the present invention.

### EXAMPLE III

### NK Cell Lytic Granules are Highly Motile at the Immunological Synapse and Require F-Actin for Post-Degranulation Persistence

NK cells are the cytotoxic effectors of the innate immune system and detect virally infected, tumorigenic or otherwise stressed cells with germline encoded activating receptors. Upon encountering a susceptible target, NK cells can mediate directed cytotoxicity following the formation of an IS and exocytosis of specialized secretory lysosomes containing lytic effector molecules such as perforinand granzyme (Orange, J. S. (2008) Nat. Rev. Immunol., 8: 713-725). The steps leading to NK cell granule exocytosis are highly regulated, as human NK cells are pre-armed with constitutively mature lytic granules and need not undergo further activation or expansion in order to kill (Wulfing et al. (2003) Proc. Natl. Acad. Sci., 100:7767-7772; Zarcone et al. (1987) Blood 69:1725-1736).

NK cell lytic granule exocytosis is preceded by the dynein-dependent convergence of granules to the microtubule organizing center (MTOC) and subsequent polarization of the MTOC and granules to the IS (Mentlik et al. (2010) Mol. Biol. Cell, 21:2241-2256). Once polarized, lytic granules undergo docking and fusion, after which their contents can be released upon the target cell. The delivery of granules from the MTOC to the plasma membrane for exocytosis is a process that is incompletely understood. A dynamic actin cytoskeleton is required for multiple aspects of cytotoxicity, including lytic granule polarization and degranulation (Rak et al. (2011) PLoS Biol., 9: e1001151; Orange et al. (2003) Proc. Natl. Acad. Sci., 100:14151-14156). Furthermore, the association of granules with actin filaments in a pervasive actin network suggests a role for actin specifically in granule trafficking immediately prior to exocytosis (Rak et al. (2011) PLoS Biol., 9: e1001151; Brown et al. (2011) PLoS Biol., 9: e1001152; Mace et al. (2012) Communicative & Integrative Biol., 5:1). The actin motor protein myosin IIA, which is also required for degranulation, is found both at the immunological synapse and the surface of lytic granules, and inhibition or loss of myosin IIA function results in impaired delivery and movement of granules at the plasma membrane (Sanborn et al. (2009) J. Immunol., 182:6969-6984; Andzelm et al. (2007) J. Exp. Med., 204: 2285-2291).

In order to address the question of lytic granule delivery and the role of the cytoskeleton in this process, the behavior of granules at the plasma membrane of activated human NK cells was determined. Total internal reflection microscopy (TIRFm) was employed to study only those granules present at the plasma membrane in combination with use of LysoTracker® red and a LAMP1-phlourin fluorescent reporter to identify degranulation events. Use of these technologies allows the identification and tracking of individual granules both before and after exocytosis. It was found that individual granules underwent dynamic, undirected movement at the plasma membrane prior to, but not following, fusion and release of granule contents. Surprisingly, depolymerization of the actin cytoskeleton with Latrunculin A (LatA) did not affect pre-exocytosis movement. The integrity of the actin cytoskeleton, however, was required for persistence of granules following fusion, demonstrating a specific interplay with and role for synaptic actin in NK cell degranulation.

### Materials and Methods

### Cell Lines

The NK92 phlourin-LAMP1 cell line (Rak et al. (2011) PLoS Biol., 9:e1001151) and YTS GFP-actin (Orange et al. (2011) J. Clin. Invest., 121:1535-1548) cell lines were generated previously. All NK cell and 721.221 and K562 target cell lines were maintained as described (Banerjee et al. (2007) J. Exp. Med., 204:2305-2320).

### Live Cell Confocal Microscopy

For imaging of NK cells with targets, NK effector cellswere suspended in RPMI 10% FBS at a concentration of 10⁶ cells/ml and incubated with 10 μM LysoTracker® Red DND-99 at 37° for 30 minutes then washed and suspended in media. Effectors were mixed at a 2:1 ratio with targets that had been pre-incubated for 5 minutes with 5 μg/ml CellMask® Plasma Membrane Stain (Invitrogen). Conjugates were plated in Lab-Tek #1.0 borosilicate chamber slides (VWR) that had been coated with 5 μg/ml anti-CD48 (BDclone TU145) for 30 minutes at 37° then washed 3 times to adhere target cells and thus facilitate imaging. Effectors and targets were incubated for 30 minutes then SYTOX Blue was added to a final concentration of 1 μM. Conjugates were imaged in a single plane on a Zeiss Axioplan Observer Z1 fluorescence microscope with a 63×1.40 NA objective and Yokigawa CSU-10 spinning disk with excitation by 405-, 488-, and 568-and 647-nm lasers in LMM5 laser combiner unit (Spectral Applied Research). Images were acquired for 90-130 minutes at a rate of one frame per minuteusing Volocity software (PerkinElmer). Temperature was maintained at 37° using a ΔT dish heater and lid (Bioptechs).

### Live Cell TIRFm

For NK92 lytic granule motility studies, pHluorin-LAMP1 NK92 cells were suspended in dye free RPMI 10% FBS media at a concentration of 7.5×10⁵ to 10⁶ cells/ml and incubated with 100 nM LysoTracker® Red DND-99 (Invitrogen) at 37° C. for 30 minutes, washed, and resuspended in media. ΔT culture dishes (Bioptechs) were coated with 5 mg/ml anti-NKp30 and -CD18, incubated at 37° C. for 1 hour or 4 degrees overnight, washed with PBS, and pre-warmed with dye free RPMI 10% FBS media. NK92 cells were plated on ΔT culture dishes immediately prior to imaging, and temperature was maintained at 37° C. throughout the experiment using a ΔT culture dish heater and lid (Bioptechs).

Cells were imaged in a single z-plane 10-15 minutes after addition to imaging chambers through an APO N TIRFm, oil immersion 60×1.49 NA objective using an Olympus IX-81 spinning disk confocal microscope with a rear-mounted TIRF illuminator and a Hamamatsu EM CCD C9100 camera. Excitation was by 480 nm (Spectra-Physics) and 561 nm (Cobalt) diode lasers through a LMM5 laser combiner unit (Spectral Applied Research). Cell image sequences were captured in TIRF mode at the interface between the cell and the glass over 60-80 minutes at 6 frames per minute using Volocity® software (PerkinElmer). Where indicated, cells were incubated with 10 μM LatA (Sigma) following 10 minutes of pre-incubation in the imaging chamber.

### Image Analysis

For analysis of live NK cell conjugates, images were identified and cropped in Volocity® (PerkinElmer). Mean centroid to IS distance was calculated using the shortest line from the centroid of the combined lytic granule region for each timepoint to the IS as determined by Volocity® software (PerkinElmer) as described previously (Mentlik et al. (2010) Mol. Biol. Cell, 21:2241-2256). Mean fluorescent intensity of SYTOX® Blue staining was measured for each time point in Volocity® and exported to GraphPad Prism® version 5.0 (GraphPad software).

For TIRFm analysis, image sequences were analyzed using Volocity® software (PerkinElmer). LysoTracker® Red and pHluorin positive events were identified and cropped in Volocity®. Object tracks were generated over time using the “track objects manually” function and the rectangular selection tool for each event; the track length, track velocity, displacement, and displacement rate of each lytic granule were measured for both LysoTracker® Red time points and pHluorin time points, and comparatively graphed on GraphPad Prism® Version 5.0 (GraphPad software). Overlays of lytic granule tracks were generated by the plotting of X and Y coordinate values from Volocity with GraphPad Prism® for both Lysotracker® Red and pHluorin. To measure the time persistence of LysoTracker® Red lytic granules within the TIRF field, the time of LysoTracker® Red emergence on the TIRF field was subtracted from the time of pHlourin appearance or LysoTracker® Red disappearance and multiplied by 60 to obtain time persistence in seconds.

For LatA experiments, granules with both Lysotracker Red and pHluorin traces were identified and cropped in Volocity®. Objects were identified using a threshold of 3 SD above the mean field fluorescence for each timepoint to account for photobleaching using the “Find Objects Using SD Intensity” function. Objects smaller than 0.032 μm² were excluded from further analysis. Tracks were then generated using the “Track Objects” feature using the “Shortest Path” tracking model, and the track length, track velocity, displacement, displacement rate, and time span of each lytic granule tabulated and graphed using GraphPad Prism® software Version 5.0.

The sum fluorescent intensity and area of each granule was also recorded at each timepoint. Using the initial appearance of pHluorin-LAMP1 as time zero, the average sum fluorescent intensity post-degranulation was calculated and graphed for lytic granules treated with DMSO vehicle control versus those treated with LatA. For area measurements, the borders of individual granules were defined as those above 3 SD.

### Statistical Analysis

The minimum sample size of lytic granules required for evaluation in a given experiment was determined using a DSS sample size calculator, with a and b error levels of 1% (DSS Research). Statistical significance was determined by performing two sample, unpaired, two-tailed t tests or Mann Whitney U tests using GraphPad Prism® version 5.0 (GraphPad Software). Differences were considered to be significant if p<0.05 and not significant if p>0.05. All error bars shown represent SD.

### Results

Polarization of Lytic Granules to the IS Precedes their Synaptic Persistence Prior to Target Cell Death

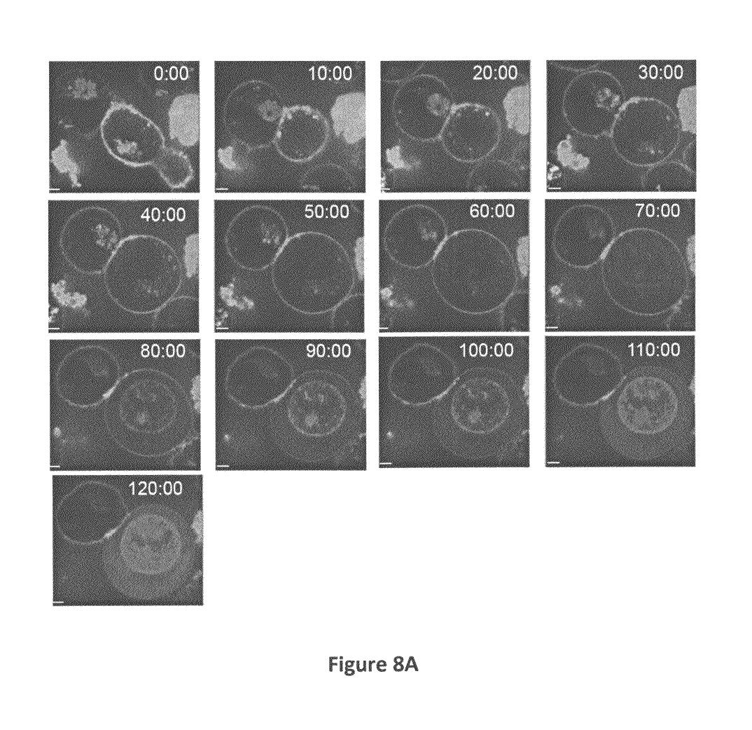

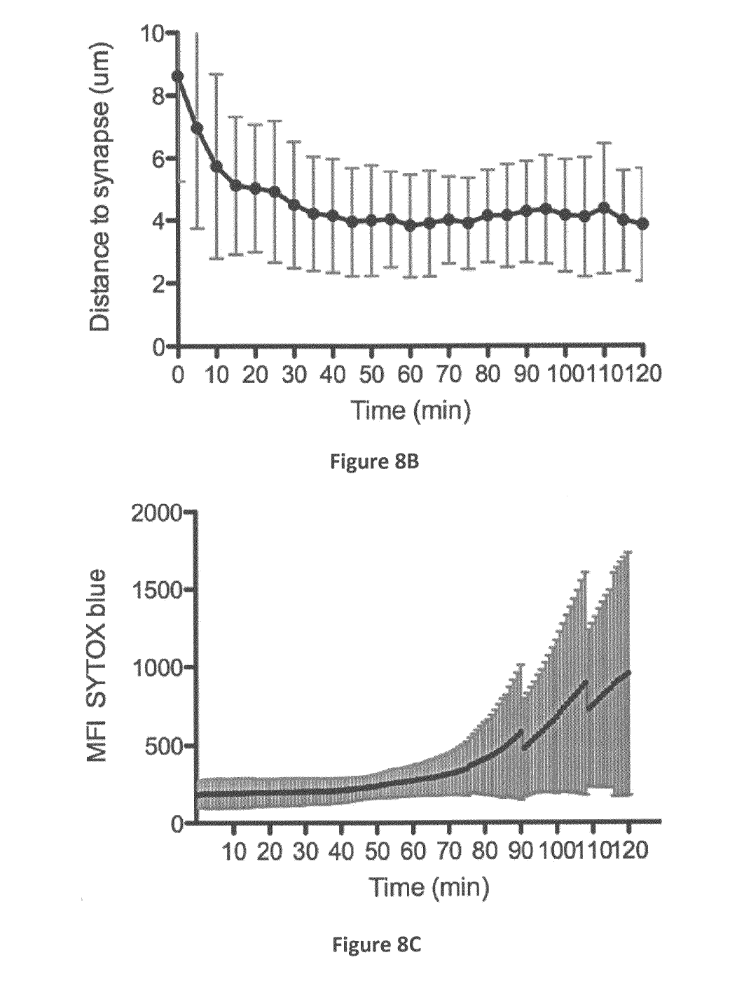

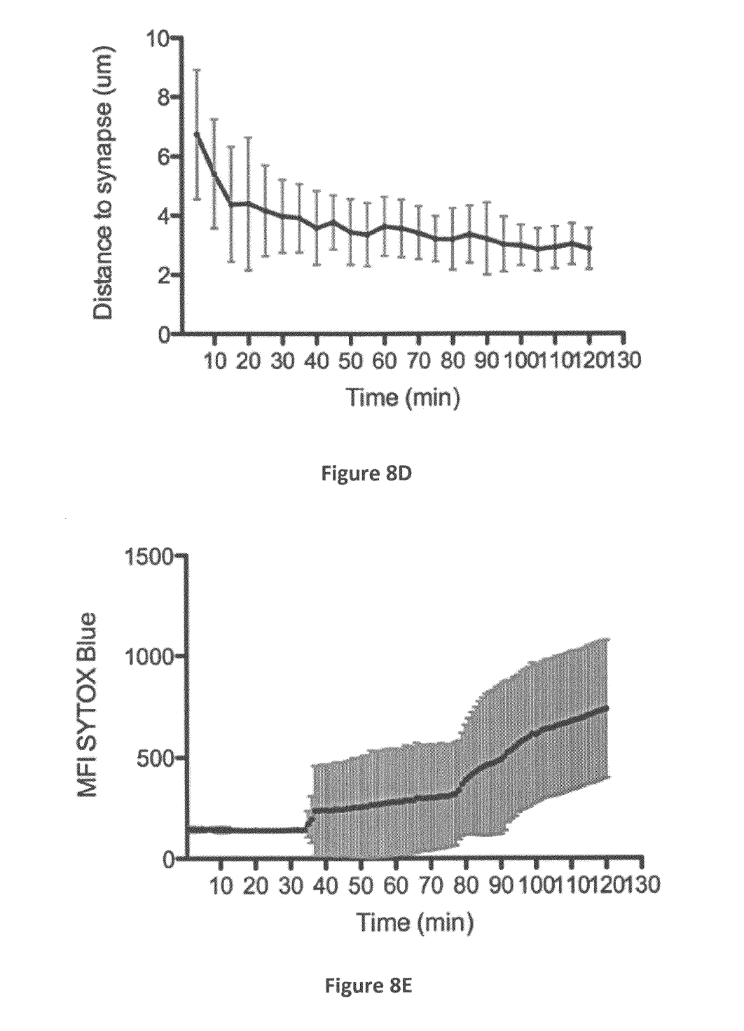

To better conceive the opportunities for control of synaptic lytic granules, the dynamics of NK cell lytic granule convergence, polarization and target cell death in live NK cells were studied. YTS GFP-actin cells were loaded with LysoTracker® Red DND-99, which selectively labels acidified organelles and fluoresces at 568 nm in acidic pH. Susceptible 721.221 targets were labeled with CellMask® to discern them from effector cells and NK-target cell conjugates were imaged every ten seconds for 60-120 minutes using live cell spinning disk confocal microscopy. Imaging was performed in the presence of SYTOX® Blue nucleic acid stain, which is impermeable to live cells and thus identifies dying cells within the population. Following initial contact with target cells, NK cell granules were rapidly converged to the MTOC as reported (Mentlik et al. (2010) Mol. Biol. Cell, 21: 2241-2256). This was followed by polarization of granules to the IS (FIG. 8A, top panel). While granule polarization occurred rapidly, however, the initiation of target cell death as marked by SYTOX® entry was not visualized until approximately a half-hour later (FIG. 8A, bottom panel). Analysis of ten conjugates supported this observation, with the mean time of granule polarization being 41.5±12 minutes (FIG. 8B) and SYTOX® Blue entry being first visualized at a mean timeof 61.5±±14 minutes (FIG. 8C). During this time granules remained converged at the MTOC and continued to be visible, suggesting they were not undergoing degranulation or recycling. Thus, granule polarization precedes initiation of target cell death by approximately 20-30 minutes.

To ensure that these observations were not specific to the YTS cell line, these same parameters were studied in a second and distinct NK cell line. NK92 GFP-tubulin NK cells conjugated to susceptible K562 targets were imaged using the same strategy as above. Similar to YTS-mediated target cell apoptosis, NK92 mediated target cell death was delayed following granule polarization. Quantitation of 6 cell conjugates confirmed this observation, with a mean time of granule polarization of 24.2 minutes (FIG. 8D), and a mean time of initiation of target cell death of 54.2 minutes (FIG. 8E). Together these results indicate that granule persistence at the IS prior to target cell death is a common feature of NK cell mediated killing.

### Lytic Granules are Dynamic Prior to, but not Following, Degranulation at the NK IS

In order to determine the behavior of lytic granules at the synapse following polarization, the NK cell immune synapse was recapitulated using activating antibodies immobilized on a glass surface, thus allowing for orientation of the NK cell synapse in the XY plane to enable high resolution imaging. NK92 cells expressing a pH sensitive GFP mutant fluorescent protein (pHlourin) fused to lysosomal activation marker protein 1 (LAMP1) were used to visualize individual degranulation events. LAMP1 (CD107a) is sorted to lytic granules and is often used as a marker of degranulation when found on the cell surface (Peters et al. (1991) J. Exp. Med., 173:1099-1109; Alter et al. (2004) J. Immunol. Methods 294:15-22). At acidic pH within lytic granules pHlourin-LAMP1 is not excited by green wavelengths, but following degranulation and exposure to neutral pH, the pHluorin exhibits fluorescence characteristics of GFP and green emission is observed (Rak et al. (2011) PLoS Biol. 9: e1001151; Miesenbock et al. (1998) Nature 394:192-195). To identify granules prior to degranulation, NK92 LAMP1-phlourin cells were loaded with LysoTracker® Red. Therefore, individual granules at the plasma membrane could be tracked over time and degranulation events observed by a transition from red fluorescence, derived from LysoTracker® red in acidified organelles, to green fluorescence derived from the neutralization of pH and subsequent activation of LAMP1-phlourin. A cross-linking antibody to the natural cytotoxicity receptor NKp30 and CD18, the β2 subunit of LFA-1, was used to activate NK cells for degranulation. Together, these signals induce polarized secretion of lytic granules in the XY plane (Rak et al. (2011) PLoS Biol., 9: e1001151; Bryceson et al. (2005) J. Exp. Med., 202:1001-1012) and allow for visualization of degranulation events by TIRFm, which provides high resolution imaging limited to the membrane proximal 100 nm of cells bound to antibody-coated glass.

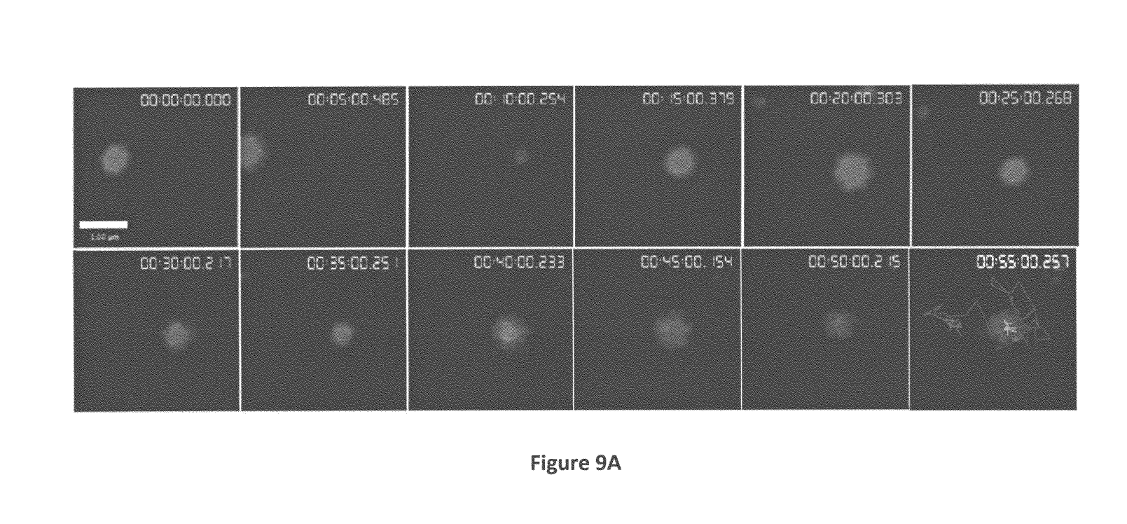

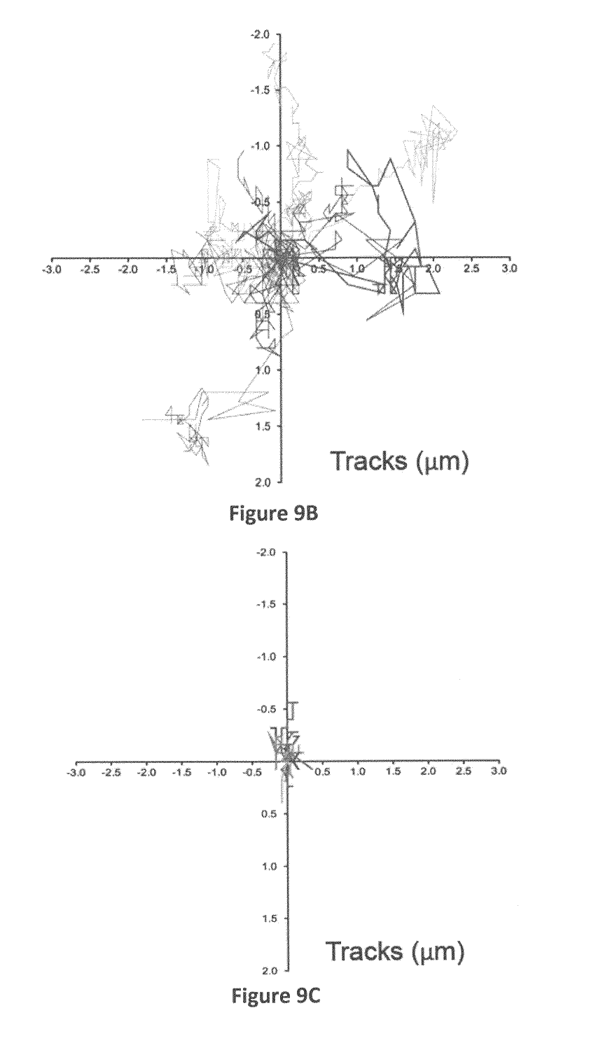

Cells were imaged using two-color TIRFm at the interface between the cell and the glass in the XY plane over timein heated imaging chambers following ten minutes of contact. Numerous polarized lytic granules, indicated by red fluorescence derived from LysoTracker® Red labeling, were identified at the plane of the IS. Over time, degranulation events were visualized at the plane of the cell cortex and observed within the TIRF field (FIG. 9A). These degranulation events were identified by a color change from red to green fluorescence.

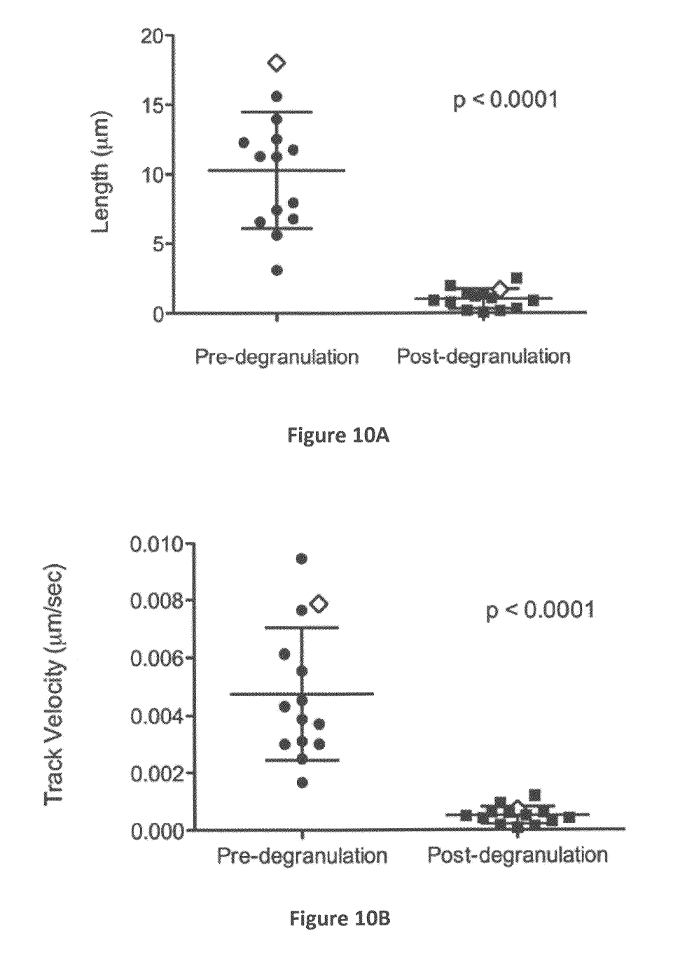

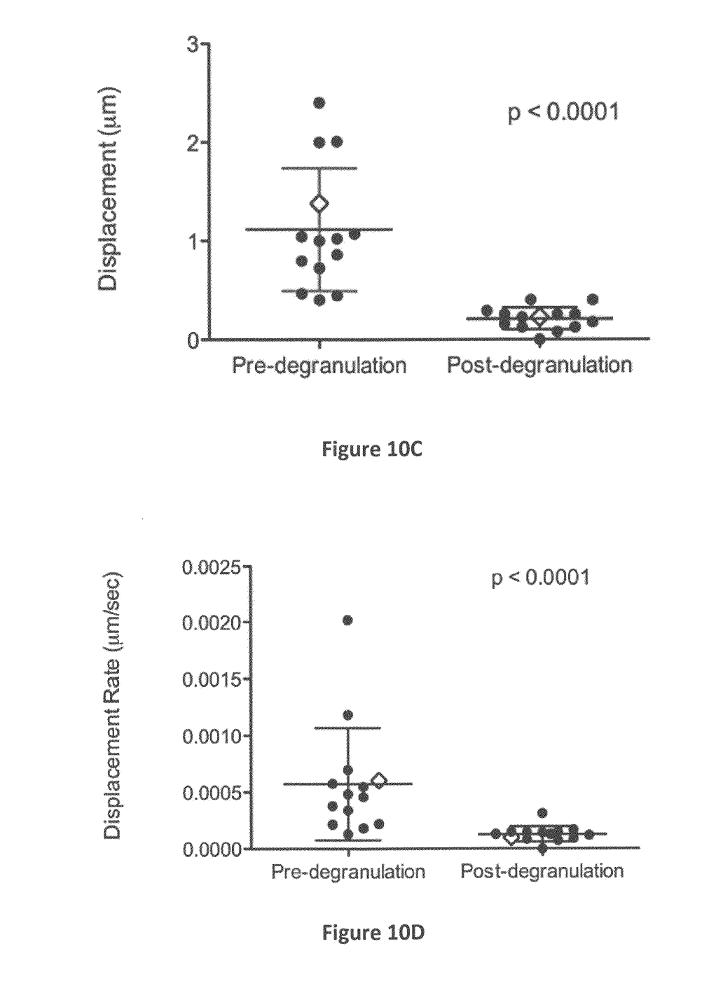

To quantify granule behavior, individual LysoTracker® Red labeled lytic granules undergoing degranulation were measured and tracked in the XY plane. Prior to degranulation, an overlay of these tracks showed seemingly random, yet highly dynamic, movement of granules navigating the cell cortex (FIG. 9B). Tracks of the same granules following degranulation, however, demonstrated greatly reduced motility as seen by shorter tracks with less displacement (FIG. 9C). This observation was further quantitated by measurement of track length, velocity, displacement and displacement rate. Strikingly, pre-degranulation granules had longer track lengths, with a mean length of 10.2±4.2 μm compared to 1.0±0.71 μm following degranulation (FIG. 10A). The mean velocity for granules pre-degranulation was 0.005 μm/sec±0.002, whereas any post-degranulation granule velocity was almost tenfold less, 0.0005±0.0003 μm/sec (FIG. 10B). In addition to track length, granules prior to degranulation had a greater displacement from their point of origin, 1.16±0.62 μm when compared to post-degranulation, 0.21±0.11 μm (FIG. 10C). Finally, the rate of this displacement in granules prior to degranulation, 0.0005±0.0005 μm/sec, was greater than that following degranulation, 0.0001±0.000067 μm/sec (FIG. 10D). Thus, lytic granules are dynamic and demonstrate considerable apparently non-directed motility at the NKIS while navigating the cell cortex before degranulation. Following degranulation, this motility is significantly reduced.

### Degranulation Events do not Predict Pre-degranulation Motility and Persistence

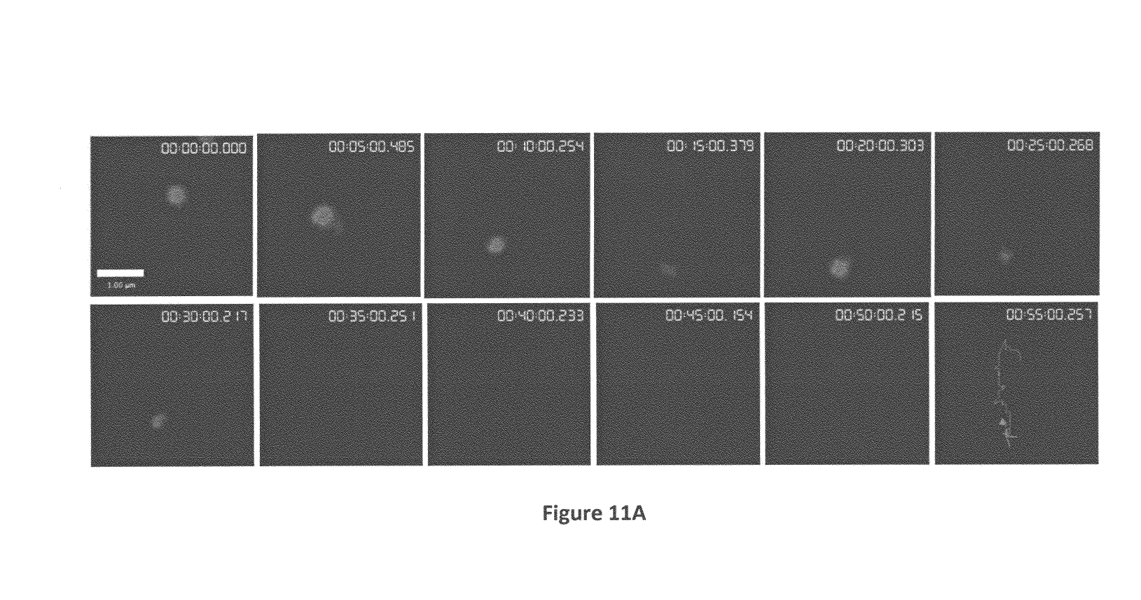

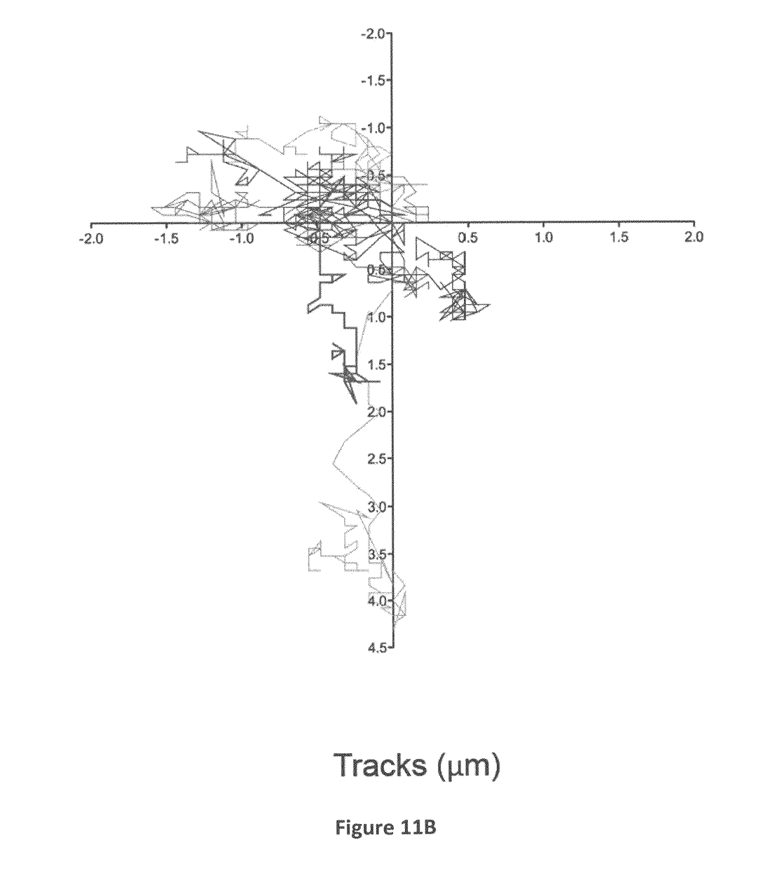

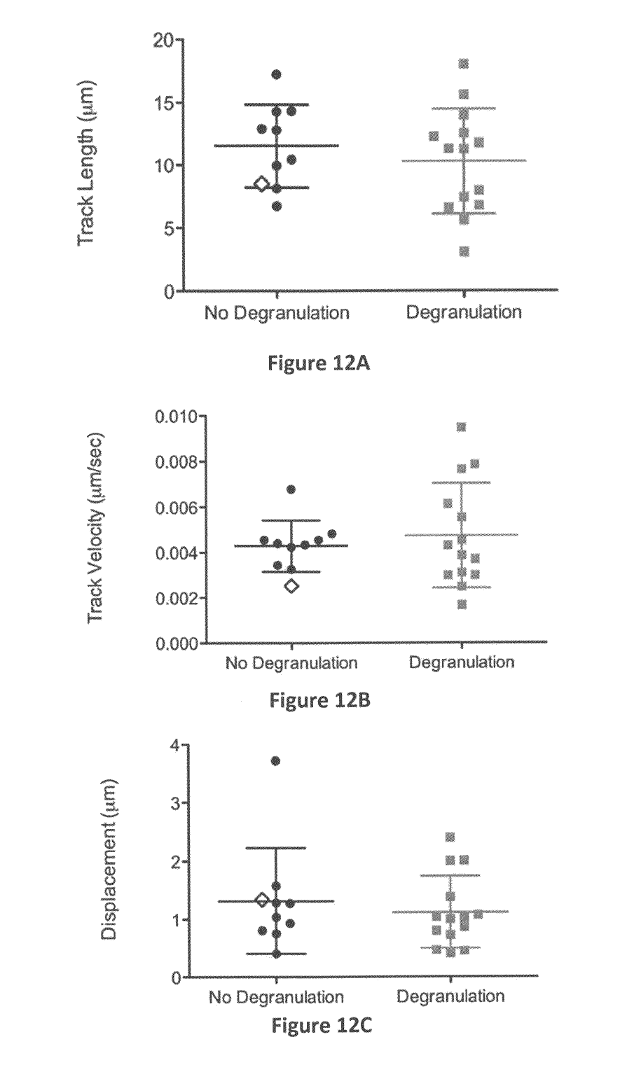

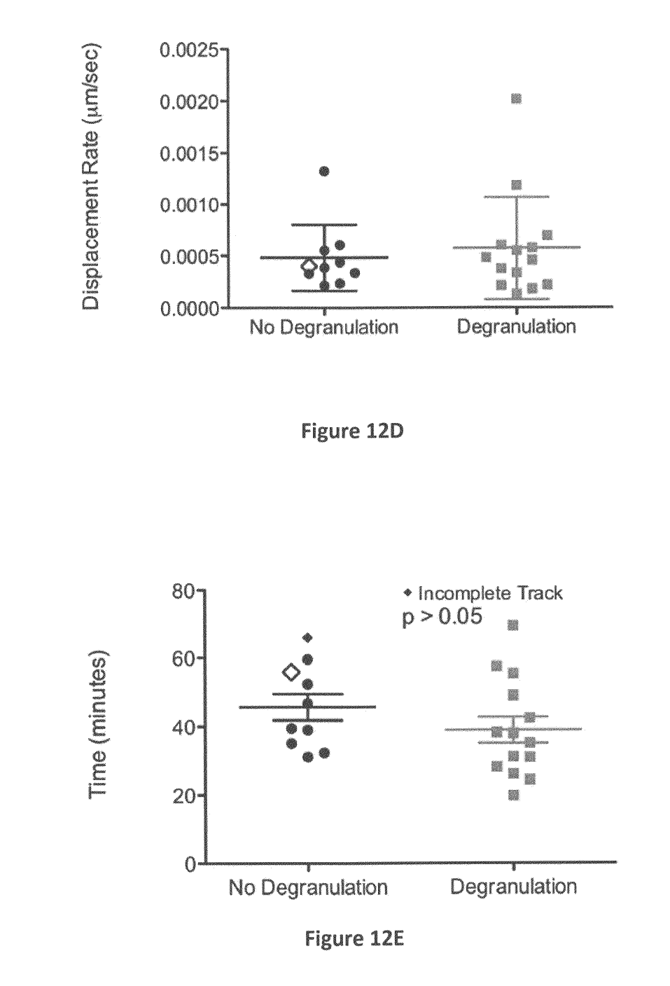

It has been observed that many more NK92 lytic granules approximate the IS than degranulate (Rak et al. (2011) PLoS Biol., 9:e1001151). As a result, a significant number of polarized granules that navigate the IS are not secreted. To investigate the dynamics of lytic granules that do not degranulate at the activated NKIS, individual LysoTracker® Red labeled lytic granules that did not result in degranulation events, and thus were distinguished by a consistent, unchanging red fluorescence color, were measured, and tracked in the XY plane throughout the duration of imaging until they disappeared from the TIRF field (FIG. 11A). Relative to LysoTracker® Red labeled NK92 granules that degranulate, those that do not degranulate were similarly dynamic. An overlay of lytic granule approximation tracks showed considerable non-directed movement (FIG. 11B). Navigating the NK cell cortex, these lytic granules had lateral mobility that was not significantly different from those that go on to degranulate. The track velocity of granules that did not degranulate was 0.004±0.001 μm/sec, which was not significantly different from the mean of those that did degranulate (FIG. 12A). Similarly the mean track length of 11.53±3.32 μm, for granules which did not degranulate, was comparable to that of ones that did (FIG. 12B). The mean displacement and displacement rate of granules that did not degranulate were 1.31±0.91 μm and 0.0004±0.0003 μm/sec respectively, which was also not significantly different from the displacement and displacement rate for those that did (FIG. 12C, 12D). Thus, irrespective of whether approximation leads to degranulation, lytic granules exhibit substantial, characteristic dynamics at the NK activating IS.

There was considerable variability in the duration of granule persistence within the synapse. Despite this, however, all of the granules that were identified and isolated, with the exception of one, maintained fluorescence for approximately 50 minutes following the start of imaging. Thus, it was reasoned that NK92 lytic granules approximate the actin-rich synapse within a defined period of time before ultimately degranulating or withdrawing from the plane of the cortex back into the cell. To determine if degranulation impacted the persistence of LysoTracker® Red labeled lytic granule tracks, the previously identified approximation events were compared. The difference between time of persistence as measured by visibility of LysoTracker® Red signal, of lytic granules that did not degranulate, 45.76±12.11 minutes, and those that did, 38.98±14.22 minutes, was not significant (FIG. 12E). Thus, lytic granules whose approximation events do not lead to degranulation have time of persistence and lateral mobility at the cortex that is statistically indistinguishable from that of those that do.

### Cortical Actin Integrity is Required for Post-Degranulation Persistence but not Pre-Degranulation Motility

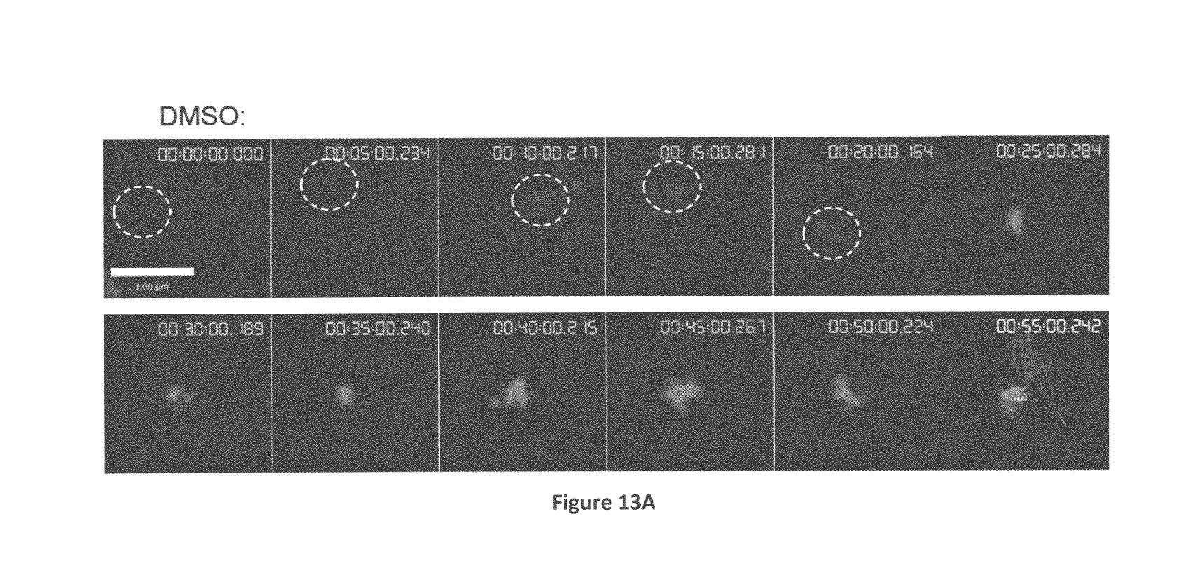

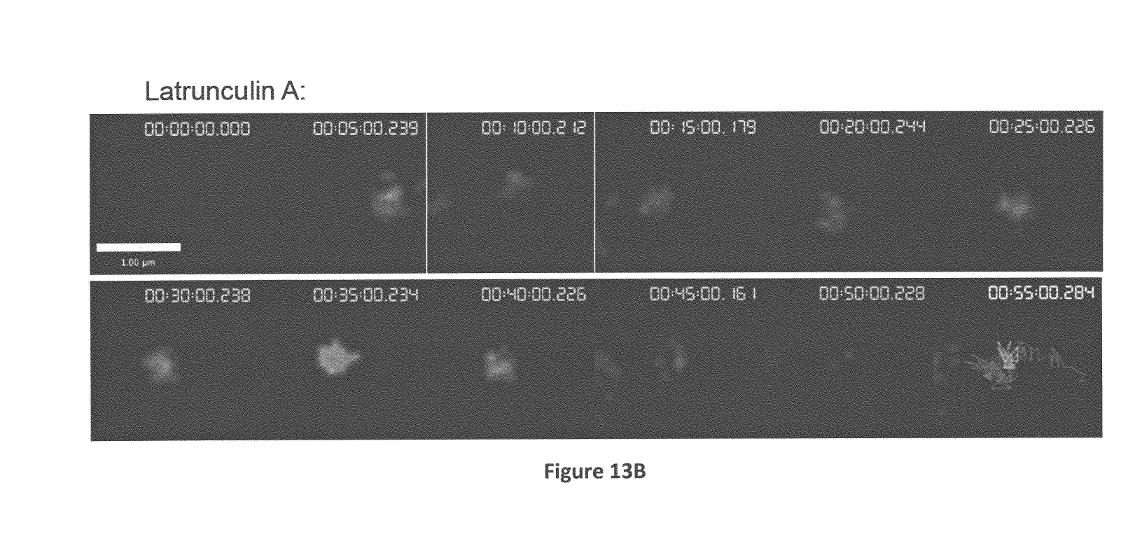

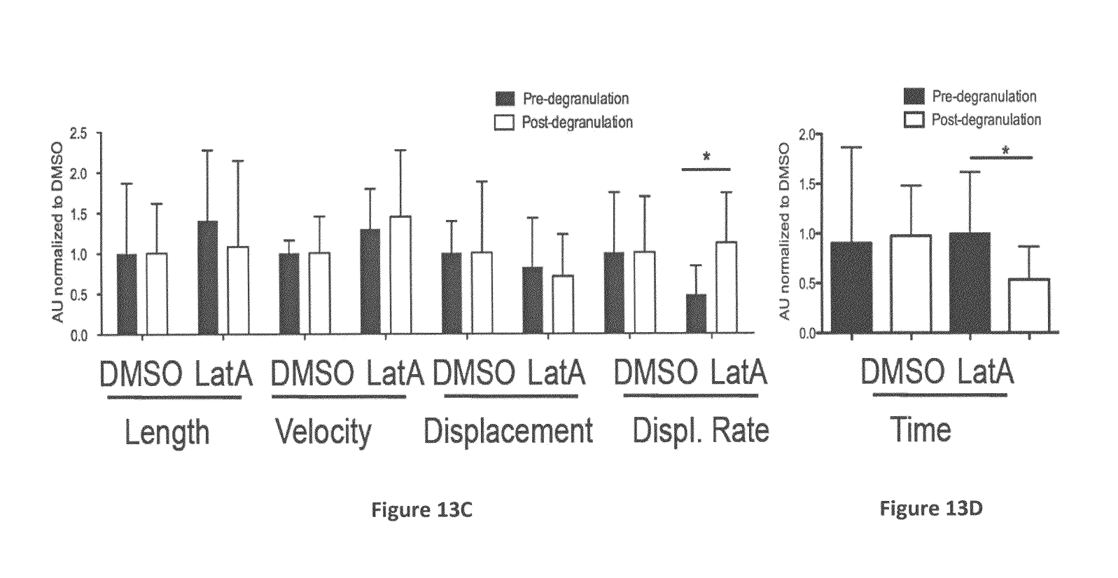

Actin cytoskeleton integrity and remodeling is required for NK cell cytotoxicity, and treatment with the actin polymerization inhibitor LatA abrogates cytotoxic function (Orange et al. (2003) Proc. Natl. Acad. Sci., 100:14151-14156; Butler et al. (2009) Curr. Biol., 19:1886-1896) and rapidly depolymerizes NK cell cortical actin filaments (Rak et al. (2011) PLoS Biol., 9: el001151). LatA was used to interrogate the role of actin dynamics in synaptic granule motility at the cortex. After 10 minutes of addition of NK92 LAMP1-pHluorin cells to an imaging chamber 10 μM LatA or vehicle control (DMSO) was added and lytic granules were observed via TIRF microscopy over time. Somewhat surprisingly, pre-degranulation motility of individual granules in LatA-treated cells was intact and was more similar than not when compared to those in control-treated cells (FIG. 13A, 13B). Despite LatA-treatment, granules demonstrated the characteristic motility prior to, followed by reduced motility after degranulation (as measured by a shift from red to green fluorescence) (FIG. 13A, 13B, final panel). Measurement of 12-18 tracks from LatA treated and control treated cells, normalized to the values to the DMSO control, demonstrated no significant differences in lytic granule track length, velocity and total displacement attributable to LatA (FIG. 13C). The only notable difference was that granules from LatA treated cells had a significantly reduced displacement rate prior to, but not following, degranulation, with a displacement rate of 0.48±0.36 μm/sec compared to the normalized control (1±0.74 μm/sec, p=0.0156).

The major difference attributed to LatA treatment, however, was that upon granule lifetime. Granules for which termination of all degranulation events were observed (as marked by the disappearance of green fluorescence) were selected and timespan was measured of the granule both pre- and post-degranulation. There was no difference in the measured lifetime of red fluorescence of Latrunculin A treated cells, 1.1±0.61 AU compared to normalized control treated cells (1.0±0.87), suggesting that time to degranulation was not affected by LatA. However, when compared to DMSO treated control cells, granules from LatA treated cells persisted for significantly less time following exocytosis, as measured by a shorter timespan of green fluorescence, 0.59±0.442 compared to control normalized to 1.0±0.56 (FIG. 13D, p=0.0441). This indicates that actin is required for the maintenance of the degranulation event once it has occurred.

### Loss of Actin Integrity Leads to Increased Granule Area

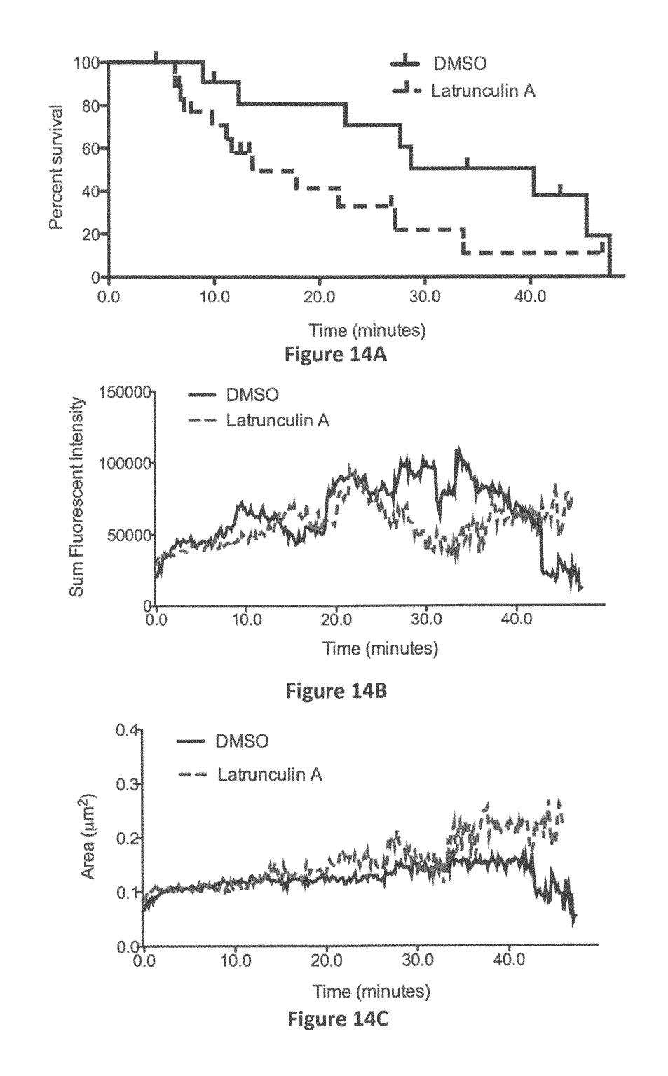

While some granules' green fluorescence persisted through the timeframe of imaging (60-80 minutes), others ceased to be visible in TIRF field following degranulation. This suggested that the “disappearing” degranulation events were withdrawn into the cell and therefore out of the field of TIRF, or that lumenal LAMP1-phlourin was diluted by diffusion throughout the membrane adjacent to the site of degranulation and thus fell below the threshold for detection by imaging. Information about the persistence of granules was plotted using a Kaplan-Meier survival curve (FIG. 14A). As imaging continued for variable times following degranulation, some imaging sequences ended with granules still visible (vertical ticks, FIG. 14A). However, compared to granules from DMSO treated cells, those from LatA treated cells demonstrated shortened survival, indicating decreased persistence following degranulation, with a median time of 13.7 minutes for 50% survival compared to 28.8 minutes for control treated cells.

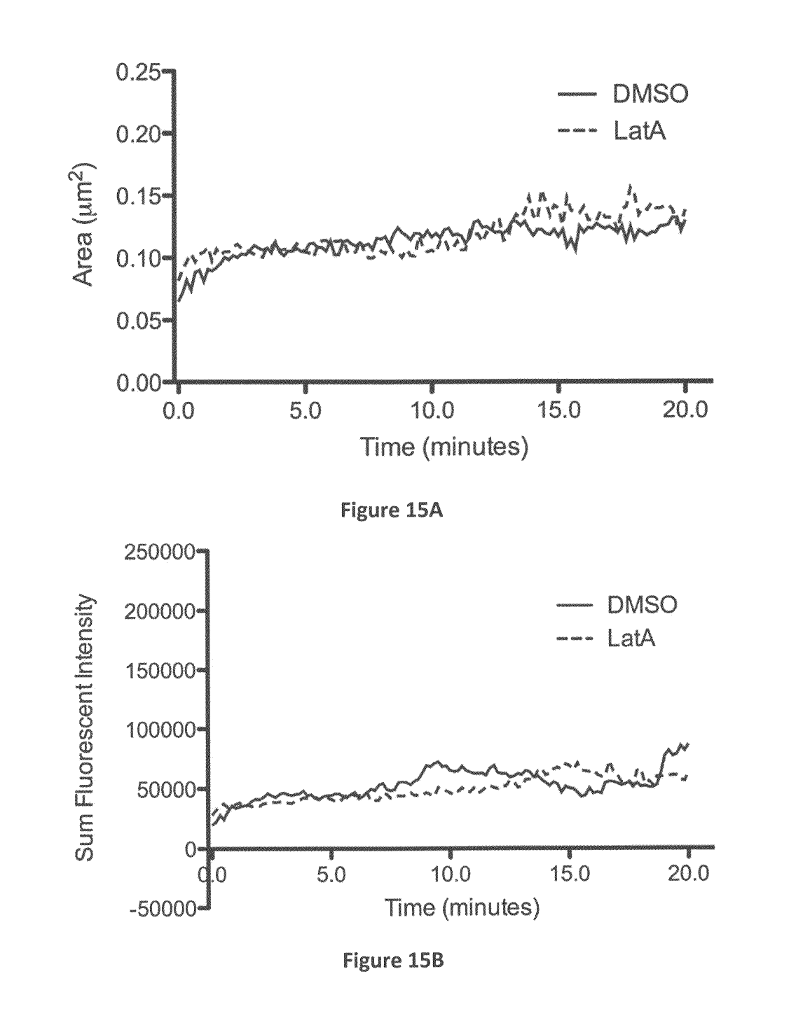

One explanation for the observed lack of persistence of granules following degranulation is that the more rapid loss of green fluorescence following degranulation in LatA-treated cells is due to faster dissipation of granule contents and/or the granule membrane. Alternatively, it may indicate a role in actin in maintaining a granule at the cortex prior to its withdrawal into the cell and thus out of the TIRF field. To help discriminate between these two models, the area and mean fluorescent intensity (MFI) of LatA and DMSO treated granules were measured at 10-second intervals following degranulation. There was a significantly greater sum fluorescent intensity (MFI*area) of granules from DMSO treated cells up to forty minutes post-degranulation at almost all time points tested (FIG. 14B, p<0.0001 by Mann Whitney U test). This was in spite of a slightly greater area of granules from LatA treated cells, as seen in FIG. 14C. This difference in area was significant by Mann Whitney U test over 40 minutes (p<0.0001), however this may have been partially due to fewer granules remaining, thus weighting the results in favor of the few remaining larger granules. Truncation of the measurements at an earlier time (20 minutes post degranulation) resulted in loss of significance in granule area, suggesting the difference may not be physiologically relevant (p=0.4438, FIG. 15A). Interestingly, truncation at 20 minutes did not affect significance of the difference in sum fluorescent intensity (p<0.0001, FIG. 15B), suggesting that over all timepoints measured granules from control treated cells had a greater sum fluorescent intensity, likely attributable to an increase in mean fluorescent intensity. Given that the intensity over a unit area is a predicted feature of the number of pHluorin molecules from the granule membrane present in the zone of TIRF illumination, it likely that the depolymerization of actin is preventing the compression of the lytic granule into the NK cell membrane. Thus, the role of actin in degranulation after the granule is delivered is predicted to be one of fully extruding granule contents.

In this study, the behaviors of lytic granules from NK cells have been determined using pH sensors in combination with TIRFm. It has been shown that NK cell granules undergo undirected yet highly dynamic motility prior to, but not following, degranulation. Actin dynamics are required not for granule motility at the cell cortex immediately preceding degranulation, but instead for the persistence of granules following it.

A longstanding paradigm for granule secretion in both NK cells and CTLs has been that granules are secreted through a central clearance, devoid of actin, and likely delivered directly by the MTOC to this clearance (Orange et al. (2003) Proc. Natl. Acad. Sci., 100:14151-14156; Stinchcombe et al. (2006) Nature 443: 462-465). However, recently in NK cells this paradigm has been challenged by the observation that a filamentous actin meshwork is present throughout the activating synapse and that granules are secreted through minimally permissive actin hypodensities (Rak et al. (2011) PLoS Biol., 9:e1001151; Brown et al. (2011) PLoS Biol., 9:e1001152). While NK cell granules are secreted centrally (Brown et al. (2011) PLoS Biol., 9:e1001152), in NK92 and ex vivo human NK cells granules have also been observed being exocytosed throughout the synapse (Rak et al. (2011) PLoS Biol., 9:e1001151). In CTL, while granules are delivered to the plasma membrane in a central area, they are also observed to travel at the cell cortex from the periphery under conditions of low affinity peptide-MHC interaction (Beal et al. (2009) Immunity 31:632-642). Taken together, these results indicate that granules are not simply centrally ejected directly from the MTOC, but may also undergo movement at the plasma membrane prior to exocytosis. The initial observation of a delay between granule polarization and target cell death (FIG. 8), led to the investigation of the dynamics of granules at the plasma membrane during this time, revealing highly dynamic, apparently undirected movement of granules prior to exocytosis.

The mechanism responsible for lytic granule movement after they have been delivered to the synapse in NK cells is unknown. It was investigated whether actin remodeling at this relatively late timepoint after synapse formation was a requirement for granule motility. In resting human NK cells, treatment with Latrunculin B arrests those granules constitutively found at the plasma membrane, suggesting actin dependent movement (Liu et al. (2010) PLoS One 5:e12870). Myosin IIA is enriched on lytic granules and enables the interaction of granules with actin, and inhibition of myosin light kinase reduces the penetration into the cortex as well as the motility of granules existing within the cortex, again suggesting actin-dependent movement of cortical granules (Sanborn et al. (2009) J. Immunol., 182:6969-6984). However, surprisingly, it is shown herein that LatA treatment (which completely depolymerizes cortical actin (Rak et al. (2011) PLoS Biol., 9:e1001151)) did not significantly affect granule movement prior to degranulation, as the velocity and displacement of granules was comparable between control- and LatA-treated cells. Granule movement at the cortex may be mediated by microtubules. Granules move on microtubules in a dynein-dependent manner prior to MTOC polarization (Mentlik et al. (2010) Mol. Biol. Cell, 21:2241-2256). In CTL, granules move in a plus-ended, kinesin-mediated direction and kinesin-1 is required to enable delivery of granules from the MTOC to the plasma membrane following MTOC polarization (Burkhardt et al. (1993) J. Cell. Sci., 104:151-162; Kurowska et al. (2012) Blood 119:3879-3889). In addition, treatment of resting human NK cells with nocodazole arrests the movement of granules at the cortex (Liu et al. (2010) PLoS One 5:e12870). The results in activated NK cells, taken together with those previously reported in otherwise resting NK cells (Liu et al. (2010) PLoS One 5:e12870), indicate that NK cells may employ multiple mechanisms of granule movement, including activation-specific, actin-independent movement. Interestingly, in neutrophils, disruption of actin by cytochalasin D treatment does not prevent movement of granules in the TIRF plane at the membrane, but instead seems to promote the accumulation of granules at the exocytic zone. This indicates that actin remodeling is not required for lateral movement, but instead may be required for clearing the way for granules through cortical actin (Johnson et al. (2012) Mol. Biol. Cell, 23:1902-1916). This may reflect commonality between cells of the innate immune system and their mechanism of secretion, although Latrunculin B treatment does not result in increased numbers of granules at the plasma membrane in resting human NK cells (Liu et al. (2010) PLoS One 5:e12870), and gross accumulation of granules was not observed at the activated NK IS in the presence of Latrunculin A.

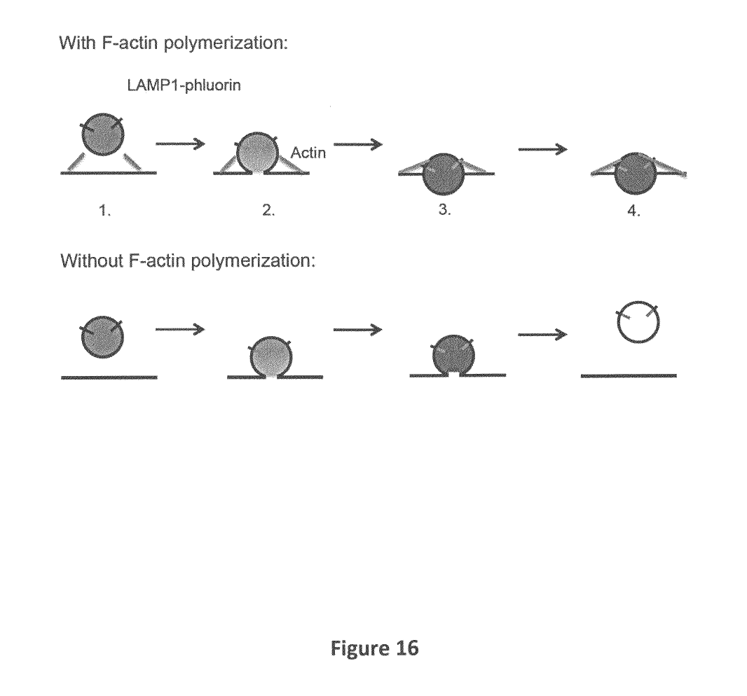

While not required for granule movement prior to exocytosis, however, it is demonstrated herein that actin dynamics affect the post-degranulation process. Specifically, actin is required for the persistence of granules at the membrane following degranulation. This is consistent with a role for F-actin in the expulsion of granule contents, the tethering of granules at the cortex, or the retention of granule contents at the plasma membrane to prevent diffusion. The data indicate the former models (FIG. 16). While there was some difference in the area of granules from LatA treated cells at later timepoints, which could support a role for actin in the prevention of lateral diffusion, this may have been due to the bias of the measurement of a few granules that had survived to this time. At all times, however, there was a significant difference in fluorescent intensity of granules following degranulation from LatA-treated cells that did not correspond to an increase in area. This indicates that actin is not required to simply act as a fence preventing the diffusion of granule contents, but may serve as a platform for the generation of force resulting in the squeezing of contents from the granules well as act as a tether to retain the granule at the cortex. This model is consistent with other cell types in which actin has been considered to be both a barrier to, and permissive of, granule exocytosis. In PC12 chromaffin cells, treatment with LatA results in a more rapid expulsion of granule contents, which would be consistent with the observation of shorter persistence of fluorescence post-degranulation (Wang et al. (2011) PLoS One 6:e29162). Together, these results indicate a requirement for force in fully extracting lytic components from granules, which contain a dense core including extracellular matrix components such as proteoglycans (Burkhardt et al. (1989) Proc. Natl. Acad. Sci., 86: 7128-7132; MacDermott et al. (1985) J. Exp. Med., 162:1771-1787). It should also be noted that, while a difference in granule velocity or displacement was observed, a significant difference between control and LatA-treated cells in displacement rate was seen, with LatA-treated cells having a significantly lower displacement rate pre-degranulation. This observation also supports a model in which actin may function in the tethering or catching of granules. Finally, actin remodeling may be required for the formation of permissive clearances in the cortex. However, while not quantified here, there was no observable defect in the number of granules that reached the TIRF plane in LatA-treated cells, consistent with prior reports (Liu et al. (2010) PLoS One 5:e12870).

The observation that granules undergo dynamic motility for such a substantive amount of time is somewhat surprising. The seemingly undirected nature of the movement may reflect a need for the granule to travel to a point of suitable actin clearance in order to be secreted. It may also indicate a requirement for granules to seek docking domains for membrane tethering and fusion. Taken together, however, the instant results demonstrated a previously unreported dynamic motility of NK cell granules prior to, but not following, degranulation. They also show a surprising role of actin dynamics in post-degranulation, but not pre-degranulation, granule behavior at the activating NK cell synapse. This indicates that in addition to serving a role early after synapse formation in enabling NK cell lytic granule localization towards the target cell, the synaptic actin meshwork allows for the persistence and compression of lytic granules. This would define a role for actin in the optimal extrusion of lytic granule contents by serving as a platform for the generation of force by which the granule contents are fully emptied.

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

1. An isolated degranulation indicator fusion protein comprising:
a) a pH sensitive detectable agent;
b) a transmembrane domain; and
c) a lysosome targeting moiety.

2. The isolated degranulation indicator fusion protein of claim 1, further comprising an endoplasmic reticulum targeting signal sequence.

3. The isolated degranulation indicator fusion protein of claim 1, further comprising a linker domain between said pH sensitive detectable agent and said transmembrane domain.

4. The isolated degranulation indicator fusion protein of claim 2, wherein said endoplasmic reticulum targeting signal sequence is the endoplasmic reticulum targeting signal sequence of the IL-2 receptor α chain.

5. The isolated degranulation indicator fusion protein of claim 1, wherein said transmembrane domain is the transmembrane domain of the IL-2 receptor α chain.

6. The isolated degranulation indicator fusion protein of claim 1, wherein said lysosome targeting moiety is the cytoplasmic tail of the lysosomal-associated membrane protein 1 (LAMP1).

7. The isolated degranulation indicator fusion protein of claim 1, wherein said pH sensitive detectable agent is a fluorescent protein.

8. The isolated degranulation indicator fusion protein of claim 7, wherein said fluorescent protein is pHluorin.

9. The isolated degranulation indicator fusion protein of claim 3, wherein said linker has the formula (G₄S)ₓ, wherein x is from about 2 to about 5 (SEQ ID NO: 5).

10. The isolated degranulation indicator fusion protein of claim 1 comprising an amino acid sequence with at least 90% identity with SEQ ID NO: 2.

11. The isolated degranulation indicator fusion protein of claim 10, which is SEQ ID NO: 2.

12. A nucleic acid molecule encoding the degranulation indicator fusion protein of claim 1.

13. The nucleic acid molecule of claim 11 having at least 90% identity with SEQ ID NO: 1.

14. The nucleic acid sequence of claim 13 which is SEQ ID NO: 1.

15. A vector comprising the nucleic acid molecule of claim 12.

16. A recombinant cell expressing the degranulation indicator fusion protein of claim 1.

17. The recombinant cell of claim 16, which is a lymphocyte.

18. The recombinant cell of claim 17, which is a natural killer cell.

19. A method for identifying an agent which modulates the degranulation process comprising,
a) contacting the cells of claim 16 with at least one agent; and
b) monitoring the signal from the pH sensitive detectable agent of the degranulation indicator fusion protein,
wherein a change in the signal from the pH sensitive detectable agent in cells contacted with said agent compared to the signal from cells which were not contacted with the agent, indicates that said agent is a modulator the degranulation process.

20. The method of claim 19, further comprising activating the cells of step a).


SEQUENCE LISTING (5 sequences)

SEQ ID NO: 1 - DNA, 957 bp, Artificial Sequence
  Synthetic sequence
atggattcatacctgctgatgtggggactgctcacgttcatcatggtgcctggctgccaggcactcgagatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttaacttatggtgttcaatgcttttcaagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataacgatcaccaggtgtacatcatggcagacaaacaaaagaatggaatcaaagctaacttcaaaattagacacaacattgaagatggaggcgttcaactagcagaccattatcaacaaaatactccaattggcgatgggcccgtccttttaccagacaaccattacctgtttacaacttctactctttcgaaagatcccaacgaaaagagagaccacatggtccttcttgagtttgtaacagctgctgggattacacatggcatggatgaactatacaaagcggccgcagctgctgctggtggtggtggttctggtggtggtggttctggtggtggtggttctgatctccaggtagcagtggccggctgtgttttcctgctgatcagcgtcctcctcctgagtgggctcacctggcagaggaagaggagtcacgcaggctaccagactatctag

SEQ ID NO: 2 - protein, 318 aa, Artificial Sequence
  Synthetic sequence
MDSYLLMWGLLTFIMVPGCQALEMSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTIFFKDDGNYKTRAEVKFEGDTLVNRIELKGIDFKEDGNILGHKLEYNYNDHQVYIMADKQKNGIKANFKIRHNIEDGGVQLADHYQQNTPIGDGPVLLPDNHYLFTTSTLSKDPNEKRDHMVLLEFVTAAGITHGMDELYKAAAAAAGGGGSGGGGSGGGGSDLQVAVAGCVFLLISVLLLSGLTWQRKRSHAGYQTI

SEQ ID NO: 3 - protein, 238 aa, Artificial Sequence
  Synthetic sequence
MSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTIFFKDDGNYKTRAEVKFEGDTLVNRIELKGIDFKEDGNILGHKLEYNYNDHQVYIMADKQKNGIKANFKIRHNIEDGGVQLADHYQQNTPIGDGPVLLPDNHYLFTTSTLSKDPNEKRDHMVLLEFVTAAGITHGMDELYK

SEQ ID NO: 4 - protein, 11 aa, Artificial Sequence
  Synthetic sequence
RKRSHAGYQTI

SEQ ID NO: 5 - protein, 25 aa, Artificial Sequence
  linker
GGGGSGGGGSXXXXXXXXXXXXXXX